(19) United States
(12) Patent Application Publication
(10) Pub. No.: US 2022/0401408 A1
(43) Pub. Date: Dec. 22, 2022

(54) COMPOSITIONS AND METHODS FOR RESCUING RETINAL AND CHOROIDAL STRUCTURE AND FUNCTION

(71) Applicants: AUCKLAND UNISERVICES LIMITED, Auckland (NZ); OCUNEXUS THERAPEUTICS, INC., San Diego, CA (US)

(72) Inventors: Colin Richard GREEN, Auckland (NZ); Nasir Mat NOR, Auckland (NZ); Monica Liliana Acosta ETCHEBARNE, Auckland (NZ); Bradford James DUFT, San Diego, CA (US)

(21) Appl. No.: 17/642,641

(22) PCT Filed: Sep. 11, 2020
(86) PCT No.: PCT/US20/50579

§ 371 Date: Mar. 11, 2022

Related U.S. Application Data
(60) Provisional application No. 62/900,379, filed on Sep. 13, 2019.
(60) Provisional application No. 62/903,504, filed on Sep. 20, 2019.

(51) Int. Cl. A61K 31/353; A61K 9/00; A61P 27/02

(52) U.S. Cl. CPC A61K 31/353; A61K 9/0019; A61P 27/02

(57) ABSTRACT

The inventions relate to the use of anti-hemichannel compounds, including anti-connexin 43 hemichannel opening compounds, to rescue or restore retinal function, to rescue or restore retinal structure, and/or to rescue or restore choroidal structure and/or function in chronic retinal and other disorders.

### RELATED APPLICATIONS

The present application claims the benefit of U.S. Provisional Patent Application No. 62/900,379, filed on Sep. 13, 2019, and U.S. Provisional Patent Application No. 62/903,504, filed on Sep. 20, 2019, both of which are incorporated herein by reference in their entirety.

### FIELD

The inventions relate generally to the retina and the choroid and other ocular processes, and to connexin hemichannels.

### INCORPORATION BY REFERENCE

All U.S. patents, U.S. patent application publications, foreign patents, foreign and PCT published applications, articles and other documents, references and publications noted herein, and all those listed as References Cited in any patent or patents that issue herefrom, are hereby incorporated by reference in their entirety. The information incorporated is as much a part of this application as if all the text and other content was repeated in the application and will be treated as part of the text and content of this application as filed.

### BACKGROUND

The following includes information that may be useful in understanding the present inventions. It is not an admission that any of the information, publications or documents specifically or implicitly referenced herein is prior art, or essential, to the presently described or claimed inventions.

Diabetes is a condition growing more and more common in which the body becomes resistant to the insulin hormone. This stops sugar, or glucose, from exiting the blood stream and entering into cells. This condition can lead to serious complications, including an eye-related disease known as diabetic retinopathy.

When a person has high blood sugar for an extended period of time, the walls of the small blood vessels throughout the body become thicker. This makes it more difficult for oxygen and important nutrients to move from the blood to the cells which rely on these nutrients and oxygen for survival. Area that are greatly affected by this include the retina, located in the back of the eye. Poor circulation in these tiny blood vessels can lead to leaking as well. When blood spills from them, it becomes stuck in the retina, decreasing its ability to translate light waves into sight. Additionally, choroidal thickness is altered in diabetes and may be related to the severity of retinopathy. The presence of diabetic macular edema is associated with a significant decrease in the choroidal thickness. See Regatieri C V, Branchini L, Carmody J., Fujimoto J G, Duker J S, Choroidal thickness in patients with diabetic retinopathy analyzed by spectral-domain optical coherence tomography. Retina. 2012 March; 32(3):563-8.

As vessels become damaged due to diabetes, the overall condition of the retina decreases drastically. Leaked blood blocks the retina while decreased nutrients and oxygen cause its tissues to die. Without treatment, the end result is decreasing vision until the individual eventually loses all sight.

It is estimated that, after twenty years of having diabetes, most people will have some signs of mild diabetic retinopathy. The pathologic process in diabetic retinopathy involves microaneurysms and punctate hemorrhages in the retina. Tiny swollen blood vessels and/or bleeding in the underlying choroid damage the receptor cells and retinal neurons and can result in blindness.

The disease of diabetic retinopathy typically progresses through a series of four stages, according to the National Eye Institute (NEI). (1) Mild non-proliferative retinopathy: This stage involves small areas of swelling in the retinal blood vessels, called microaneurysms. (2) Moderate non-proliferative retinopathy: As the disease progresses, an eye doctor may now be able to see visible swelling and distortion of the retinal blood vessels. They may also lose their ability to transport oxygen and nutrients at this stage. (3) Severe non-proliferative retinopathy: This stage sees worsening of vessel blockages, depriving parts of the retina of blood. New blood vessels may also grow, blocking areas of the retina. (4) Proliferative diabetic retinopathy (PDR): Finally, these newly growing blood vessels proliferate inside of the retina, leading to leakage, vision loss, and scar tissue that can lead to retinal detachment and blindness.

Diabetic retinopathy is mainly treated in two ways: injections and laser surgery. Injections involve putting a medication such as a corticosteroid or a vascular endothelial growth factor (VEGF) antagonist directly into the eye. Surgically, doctors can use lasers to burn parts of the retina. By effectively killing these areas, the limited blood supply available can go to the remaining live tissue, helping preserve vision.

Unfortunately, there is no known cure for diabetic retinopathy. The damage caused by blood vessel growth, leakage, and oxygen deprivation is permanent, and diabetic retinopathy is not a completely reversible condition with current treatments.

Despite being a critical part of the metabolite delivery system to the outer retina, the choroid remains poorly understood. Zouache and Luthert, The Choroid In AMD: A Critical Point of Failure? Retina Specialist Jan. 8, 2018. One of two principal blood supplies to the retina, the choroid supplies blood to the outer RPE, the photoreceptors and a few of the overlying tissue layers. Choroidal failure plays in the pathogenesis of age-related macular degeneration. Changes have been reported in the choroid in both early and late-stage AMD. Additionally, in maculae presenting basal laminar deposits, geographic atrophy and disciform scarring, the vascular density of the choriocapillaris is significantly smaller than in normal maculae. Zouache and Luthert, supra. Importantly, patients with choroidal changes are at risk of developing retinal vein occlusions. Treatments for abnormal choroidal structure and function are needed.

This patent relates to the important discovery of methods and compositions comprising anti-hemichannel compounds that can fundamentially reverse diabetic retinopathy and restore retinal and choroidal structure and function in this and other diseases, disorders and conditions.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

This patent is directed to methods and compositions for the use of anti-hemichannel compounds to restore and rescue retinal structure and function. Even single doses were found to be useful over significant periods of time. The patent is also directed to methods and compositions for the use of anti-hemichannel compounds to restore and rescue choroidal structure and function.

Data show, for example, that anti-hemichannel compounds can be used to enhance and restore the function of the retina, including in chronic retinal diseases, conditions and disorders. Amongst other things, the data show that anti-hemichannel compounds can be used to improve the function of photoreceptors and bipolar cells in the inner retina. They also show, for example, that anti-hemichannel compounds can be used to protect, enhance and restore inner retinal cells and improve inner retinal function, to improve and recover the phototransduction pathway and post-photoreceptor neuron response, and to improve and recover the the retinal layer structure. It was also discovered that anti-hemichannel compounds can preserve and enhance retinal layer structures as measured by OCT, and that choroidal structure is also improved and recovered.

The patent is also directed to methods and compositions for the use of anti-hemichannel compounds in reversing chronic ocular diseases previously believed to be intractable. The patent describes the use of anti-hemichannel compounds to not only protect and improve but rescue and restore retinal function in chronic ocular diseases, disorders and conditions where retinal and/or choroidal damage was previously thought to be fundamentally irreversible, including, for example, diabetic retinopathy, non-proliferative diabetic retinopathy (NEI stages 1, 2 and/or 3, designated “mild,” “moderate” and “severe” non-proliferative retinopathy), diabetic macular edema, inflammatory or infectious chroiditis, uveitis, age-related macular degeneration (wet and dry), geographic atrophy, and other chronic disorders of the retina characterized in whole or in part by loss of retinal structure and/or function.

The patent also describes the use of anti-hemichannel compounds to treat disorders of the choroid characterized in whole or in part by loss of choroidal structure and/or function. The methods, compounds and compositions of the invention can be used to not only protect and improve but rescue and restore choroidal structure and/or function.

The patent also describes the use of orally-delivered anti-hemichannel compounds for restoring retinal function in afflicted patients, the use of orally-delivered anti-hemichannel compounds for and reversing or substantially reversing chronic retinal disease.

The patent also describes the use of orally-delivered anti-hemichannel compounds for rescuing retinal function in patients in need suffering from chronic ocular disease. The patent also describes the use of orally-delivered anti-hemichannel compounds for rescuing retinal structure in patients in need suffering from chronic ocular disease.

The patent also describes the use of orally-delivered anti-hemichannel compounds for rescuing choroid structure and function in patients in need thereof.

The patent is also directed, in another aspect, to the use of anti-hemichannel compounds to protect against diabetic retinopathy occurring secondary to spontaneous and chronic systemic hyperglycemia, and to reverse the diabetic retinopathy that may exist.

The patent is also directed to methods for the use of anti-hemichannel compounds for these purposes, including, for example, tonabersat, a benzopyran compound (cis-6-acetyl-4S-(3-chloro-4-fluoro-benzoylamino)-3,4-dihydro-2,2-dimethyl-2H-benzo[b]pyrane-3 S-ol (SB-220453, also referred to as Xiflam or tonabersat).

The inventions relate, in one aspect, for example, to the use of anti-hemichannel compounds to reverse retinal and choroidal damage in a subject with diabetes or other conditions characterized in whole or in part by loss of retinal and/or chorodial structure and/or function.

This patent describes, in one aspect, the use of compounds and methods to modulate connexin hemichannels, including connexin 43 hemichannels, to rescue or restore retinal function. It also describes the use of compounds and methods to modulate connexin hemichannels, including connexin 43 hemichannels, to rescue or restore retinal structure.

This patent describes, in one aspect, the use of compounds and methods to modulate connexin hemichannels, including connexin 43 hemichannels, to rescue or restore choroidal function. It also describes the use of compounds and methods to modulate connexin hemichannels, including connexin 43 hemichannels, to rescue or restore choroidal structure.

It also describes, in another aspect, by way of example, the use of anti-hemichannel compounds, including anti-connexin 43 hemichannel opening compounds, to preserve choroidal structure and function, to preserve retinal structure and function, to restore retinal function, to rescue retinal function, and to protect against and reverse diabetic retinopathy occurring secondary to spontaneous and chronic systemic hyperglycemia.

Methods of the invention are useful to rescue and restore choroidal structure and function, to restore retinal function, to rescue retinal function, and to protect against and reverse diabetic retinopathy and macular edema occurring secondary to spontaneous and chronic systemic hyperglycemia in a subject by administration of an anti-hemichannel compound to a subject who would benefit therefrom, as well as in other chronic retinal disorders referenced herein.

It is another object of the invention to provide compounds, compositions, formulations, kits, doses and methods for the treatment of diseases, disorders and conditions that will benefit from restoration or rescue of retinal structure, rescue of retinal function, and/or restoration of retinal function.

It is another object of the invention to provide compounds, compositions, formulations, kits, doses and methods for the treatment of diseases, disorders and conditions that will benefit from restoration or rescue of choroidal structure, rescue of choroidal function, and/or restoration of choroidal function.

It is another object of the invention to provide compounds, compositions, formulations, kits and methods for the treatment of diseases, disorders and conditions that will benefit from protection against loss of retinal function.

It is another object of the invention to provide compounds, compositions, formulations, kits and methods for the treatment of diseases, disorders and conditions that will benefit from protection against loss of choroidal function.

In some aspects, the method of treatment is applied to mammals, e.g., humans.

Anti-hemichannel compounds useful in the present invention include compounds of Formula I, for example Xiflam (tonabersat), and/or a prodrug of any of the foregoing compounds, and other anti-hemichannel compounds described or incorporated by reference herein. In some embodiments, the hemichannel blocker is a small molecule other than Xiflam (tonabersat), for example, a hemichannel blocker described in Formula I or Formula II in US Pat. App. Publication No. 20160177298, filed in the name of Colin Green, et al., the disclosure of which is hereby incorporated in its entirety by this reference.

Various preferred embodiments include use of an orally available small molecule anti-hemichannel compound, to treat diseases, disorders and conditions characterized at least in part by loss of retinal and/or choroidal structure or function, or to treat subjects who are or may be at risk for loss of retinal and/or choroidal structure or function. In one embodiment, retinal and/or choroidal structure or function is restored, substantially or completely, by treating with anti-hemichannel compounds as described, including orally available anti-hemichannel compounds.

Other preferred embodiments include use of an orally available small molecule anti-hemichannel compound, to treat subjects who are or may be at risk for loss of retinal and/or choroidal structure or function.

Other aspects of the invention include methods of improving or restoring choroidal blood flow in a subject having a chronic retinal disorder, comprising administering an effective amount of a hemichannel blocker to said subject.

Other aspects of the invention include methods of improving or restoring the choroidal vascular blood flow to the outer retina in a subject having a chronic retinal disorder, comprising administering an effective amount of a hemichannel blocker to said subject.

Included are methods for increasing survival of, and rescuing or restoring, retinal function and/or choroidal function in a subject in need thereof, comprising, e.g., administering to said subject a survival-promoting amount of N-[(3S,4S)-6-acetyl-3-hydroxy-2,2-dimethyl-3,4-dihydrochromen-4-yl]-3-chloro-4-fluorobenzamide (Xiflam). In some embodiments, the the survival-promoting amount is about 10 to about 200 mg per day. In other embodiments, the survival-promoting amount is about 20 to about 100 mg per day. These amounts may be administered in single or divided doses, e.g., BID. Other daily doses, as well as particularly useful weekly, monthly and implant dosing and dosing regimens have also been discovered and are provided herein.

In some methods the increasing survival, rescuing or restoring treats a chronic retinal disorder. In other aspects, the chronic retinal disorder is diabetic retinopathy or diabetic macular edema. In other aspects the increasing survival methods treat a chronic retinal disorder selected from the group consisting of wet age-related macular degeneration, dry age-related macular degeneration, geographic atrophy and hypertensive retinopathy.

In other aspects, the methods of increasing survival, rescuing or restoring the chronic retinal disorder is caused by retinal degeneration, edema, diabetes, ischemic retinal degeneration, retinal vascular occlusion, and central retinal vein occlusion.

In other aspects of methods of the invention, mixed a-wave function and/or improved mixed b-wave function are improved or normalized.

In other aspects of methods of the invention, retinal PII and PIII rod and cone function are improved.

In other aspects of methods of the invention, retinal ERG function is improved or normalized.

In still other aspects of methods of the invention, inner retinal function is improved or normalized.

In other aspects of methods of the invention, photoreceptor function is improved or normalized.

Also included are methods for increasing survival of, rescuing or restoring retinal structure in a subject in need thereof, disorder, comprising administering to said subject 10 to 200 mg per day of N-[(3S,4S)-6-acetyl-3-hydroxy-2,2-dimethyl-3,4-dihydrochromen-4-yl]-3-chloro-4-fluorobenzamide (Xiflam), or 1.4 mg/kg per day, or other doses noted herein. In some embodiments, the retinal structure comprises retinal pigment epithelium, retinal vascular endothelium, and/or retinal layer structure. In other embodiments, micro- and/or macro-aneurysms in the retina are reduced.

Also included are methods for increasing survival of, rescuing or restoring choroidal function in a subject in need thereof, comprising administering to said subject 10 to 200 mg per day of N-[(3S,4S)-6-acetyl-3-hydroxy-2,2-dimethyl-3,4-dihydrochromen-4-yl]-3-chloro-4-fluorobenzamide (Xiflam), or other doses noted above or herein. In some embodiments of these methods, choroidal blood flow is improved or normalized. In other embodiments, choroidal vascular blood flow supplying the outer retina is improved or normalized. In still other embodiments of these methods, modulation of choroidal blood flow is improved or normalized.

Also described claimed herein are methods increasing survival of choroidal structure in a subject in need thereof, comprising administering to said subject 10 to 200 mg per day of N-[(3S,4S)-6-acetyl-3-hydroxy-2,2-dimethyl-3,4-dihydrochromen-4-yl]-3-chloro-4-fluorobenzamide (Xiflam). In In some embodiments of these methods choroidal thickness is improved. In other embodiments, the choroidal vascular bed is improved or normalized.

In certain embodiments of the invention, increasing survival of retinal function is restoring or rescuing retinal function.

In other embodiments of the invention, increasing survival of retinal structure is restoring or rescuing retinal structure.

In other embodiments of the invention, increasing survival of choroidal function is restoring or rescuing choroidal function.

In other embodiments of the invention, increasing survival of choroidal structure is restoring or rescuing choroidal structure.

In various embodiments, the small molecule that blocks or ameliorates or inhibits hemichannel opening is a prodrug of Xiflam (tonabersat) or an analog thereof.

In another aspect, the invention provides the use of a hemichannel blocker in the manufacture of a medicament for use in the treatment of subjects, or of the diseases, disorders and conditions, described or referred to herein. The medicament will comprise, consist essentially of, or consist of an anti-hemichannel compound. In one embodiment, the anti-hemichannel compound is a small molecule anti-hemichannel compound. In another embodiment, the small molecule anti-hemichannel compound an orally-available small molecule anti-hemichannel compound.

In one embodiment, the medicament will comprise, consist essentially of, or consist of a small molecule hemichannel blocker, one example of an anti-hemichannel compound. In one embodiment, the medicament will comprise, consist essentially of, or consist of a compound according to Formula I or Formula II in US Pat. App. Publication No. 20160177298. In one embodiment, the medicament will comprise, consist essentially of, or consist of Xiflam (tonabersat).

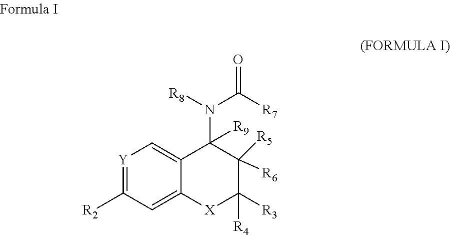

wherein Y is C—R₁;

R₁ is acetyl;

R₂ is hydrogen, C₃₋₈ cycloalkyl, C₁₋₆alkyl optionally interrupted by oxygen or substituted by hydroxy, C₁₋₆alkoxy or substituted aminocarbonyl, C₁₋₆alkylcarbonyl, C₁₋₆alkoxycarbonyl, C₁₋₆ alkylcarbonyloxy, C₁₋₆alkoxy, nitro, cyano, halo, trifluoromethyl, or CF₃S; or a group CF₃-A-, where A is —CF₂—,
—CO—, —CH₂—, CH(OH), SO₂, SO, CH₂—O, or CONH; or a group CF₂H-A′— where A′ is oxygen, sulphur, SO, SO₂, CF₂ or CFH; trifluoromethoxy, C₁₋₆alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆alkoxycarbonylamino, C₁₋₆alkyl-thiocarbonyl, C₁₋₆alkoxy-thiocarbonyl, C₁₋₆alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆alkoxysulphinylamino or C₁₋₆alkoxysulphonylamino, or ethylenyl terminally substituted by C₁₋₆alkylcarbonyl, nitro or cyano, or —C(C₁₋₆alkyl)NOH or —C(C₁₋₆alkyl)NNH₂; or amino optionally substituted by one or two C₁₋₆alkyl or by C₂₋₇ alkanoyl; one of R₃ and R₄ is hydrogen or C₁₋₄ alkyl and the other is C₁₋₄alkyl, CF₃ or CH₂X^{a} is fluoro, chloro, bromo, iodo, C₁₋₄alkoxy, hydroxy, C₁₋₄alkylcarbonyloxy, —S—C₁₋₄alkyl, nitro, amino optionally substituted by one or two C₁₋₄alkyl groups, cyano or C₁₋₄alkoxycarbonyl; or R₃ and R₄ together are C₂₋₅ polymethylene optionally substituted by C₁₋₄alkyl;
R₅ is C₁₋₆alkylcarbonyloxy, benzoyloxy, ONO₂, benzyloxy, phenyloxy or C₁₋₆alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄alkyl, cyano, azido, C₁₋₄alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C₁₋₆ alkyl amino, amino-C₁₋₆ alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆alkyl;

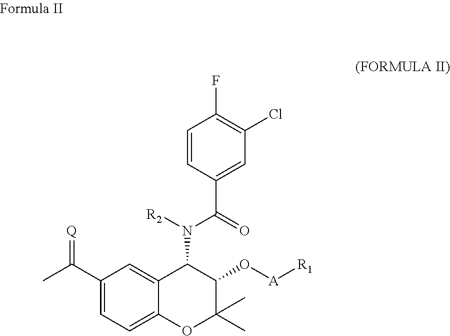

wherein
   Q is O or an oxime of formula ═NHOR₄₃, wherein R₄₃ is (i) selected from H, C₁₋₄ fluoroalkyl or optionally substituted C₁₋₄ alkyl, or (ii) -A₃₀₀-R₃₀₀, wherein A₃₀₀ is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₃₀₀, R₃ and R₄ are selected independently from H, fluoro, C₁₋₄ alkyl, or C₁₋₄ fluoroalkyl, or R₃ and R₄ together with the atom to which they are attached form a cyclopropyl group, and R₃₀₀ is selected from groups [1], [2], [2A], [3], [4], [5] or [6];
R₂ is H,
A is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₁, R₃ and R₄ are selected independently from H, fluoro, C₁₋₄ alkyl, or C₁₋₄ fluoroalkyl, or R₃ and R₄ together with the atom to which they are attached form a cyclopropyl group,
R₁ is selected from groups [1], [2], [2A], [3], [4], [5] and [6] wherein the atom marked ** is directly connected to A:

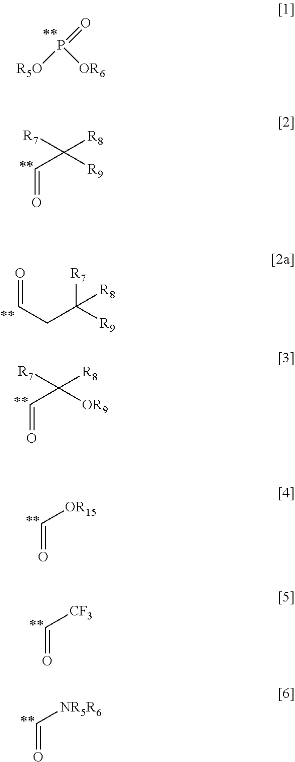

R₅ and R₆ are each independently selected from H, C₁₋₄ alkyl, C₁₋₄ fluoroalkyl, and benzyl;
   R₇ is independently selected from H, C₁₋₄ alkyl, and C₁₋₄ fluoroalkyl;
   R₈ is selected from:
   (i) H, C₁₋₄ alkyl, or C₁₋₄ fluoroalkyl, or
   (ii) the side chain of a natural or unnatural alpha-amino acid, or a peptidomimetic or other peptide as described herein, or
   (iii) biotin or chemically linked to biotin;
   R₉ is selected from H, —N(R₁₁)(R₁₂), or —N+(R₁₁)(R₁₂)(R₁₃)X⁻, or —N(R₁₁)C(O)R₁₄ wherein R₁₁, R₁₂, and R₁₃ are independently selected from H, C₁₋₄ alkyl, or C₁₋₄ fluoroalkyl,
   R₁₄ is H, C₁₋₄ alkyl, or C₁₋₄ fluoroalkyl,
   R₁₅ is independently selected from C₁₋₄ alkyl and C₁₋₄ fluoroalkyl, and
   X⁻ is a pharmaceutically acceptable anion.

The term “comprising,” which is synonymous with “including,” “containing,” or “characterized by,” is inclusive or open-ended and does not exclude additional, unrecited elements or ingredients from the medicament (or steps, in the case of a method). The phrase “consisting of” excludes any element, step, or ingredient not specified in the medicament (or steps, in the case of a method). The phrase “consisting essentially of” refers to the specified materials and those that do not materially affect the basic and novel characteristics of the medicament (or steps, in the case of a method). The basic and novel characteristics of the inventions are described throughout the specification, and include the ability of medicaments and methods of the invention to block or modulate connexin gap junction hemichannels and to preserve, protect, and restore or rescue retinal structure, to preserve, protect, and restore or rescue choroidal structure, to preserve, protect, and restore or rescue retinal function, to preserve, protect, and restore or rescue choroidal function, as the case may be. Material changes in the basic and novel characteristics of the inventions, including the medicaments and methods described herein, include an unwanted or clinically undesirable, detrimental, disadvantageous or adverse diminution of hemichannel modulation and/or preservation, protection, restoration or rescue of retinal structure, preservation, protection, restoration or rescue of choroidal structure, preservation, protection, restoration or rescue of retinal function, preservation, protection, restoration or rescue of choroidal function. In one embodiment, the medicament will comprise, consist essentially of, or consist of a connexin 43 hemichannel blocker, for example, a small molecule connexin 43 hemichannel blocker.

In another aspect, the invention provides the use of a hemichannel blocker in the manufacture of a medicament (or a package or kit containing one or more medicaments and/or containers, with or without instructions for use) for modulation of a hemichannel and treatment of any of the diseases, disorders and/or conditions described or referred to herein. In one aspect, for example, the invention provides the use of a small molecule connexin hemichannel blocker, including, for example, Xiflam and/or an analogue or prodrug thereof. In one embodiment, the medicament will comprise, consist essentially of, or consist of a connexin 43 hemichannel blocker, for example, a small molecule connexin 43 hemichannel blocker. In one embodiment, the hemichannel blocker composition useful in the invention may include a pharmaceutically acceptable carrier and may be formulated as a pill, a solution, a microsphere, a liposome, a nanoparticle, an implant (including, for example, peritoneal, subcutaneous and ocular implants, as well as slow- or controlled-release implants), a matrix, or a hydrogel formulation, for example, or may be provided in lyophilized form.

The hemichannel being modulated for the purposes described herein may be any connexin of interest for that purpose. For example, the hemichannel being modulated for the purposes described herein may be a connexin hemichannel expressed in the retina, in blood vessels, and/or in the vascular wall. In one embodiment the hemichannel blocker blocks a connexin hemichannel in a blood vessel. In other embodiments the hemichannel blocker blocks a connexin hemichannel in a blood microvessel. In other embodiments the hemichannel blocker blocks a connexin hemichannel in a capillary. In other embodiments the hemichannel blocker blocks a connexin hemichannel in endothelium.

In various embodiments, by way of example, the hemichannel being modulated comprises one or more of connexin 36 (Cx36), connexin 37 (Cx37), connexin 40 (Cx40), connexin 43 (Cx43), connexin 45 (Cx45), connexin 57 (Cx57), connexin 59 (Cx59) and/or connexin 62 (Cx62).

In one embodiment, particularly as it relates to the retina, the hemichannel being modulated comprises one or more of a Cx36, Cx37, Cx40, Cx43, Cx45 or Cx57 protein. Targeted hemichannel connexins include one or more of selected hemichannel connexins in blood vessels (e.g, Cx37, Cx40 or Cx43), as well as hemichannel connexins in astroglial cells (e.g., Cx43), amacrine cells (e.g., Cx36, Cx45), bipolar cells (e.g., Cx36, Cx45), the outer and inner plexiform layer, the ganglion cell layer (e.g., Cx36, Cx45), cone photoreceptors and retinal endothelial cells, and other retinal neurons, for example. In some embodiments, Cx36 and Cx43 hemichannels are targeted. In one particular embodiment, the hemichannel and/or hemichannel being modulated comprises Cx43. In one embodiment, hemichannels comprising connexins in the cells of the outer plexiform layer are targeted (e.g., Cx43), where methods of the invention can stop and reverse OPL thinning and rescue the OPL.

In other embodiments, particularly those relating to the choroid or blood vessels of the retina, the hemichannel being modulated may preferentially comprise one or more of a Cx37, Cx40 or Cx43 protein. In one particular embodiment, the hemichannel and/or hemichannel being modulated comprises Cx43. In one embodiment, hemichannels comprising vessel connexins in cells of the outer choroid, also known as Haller's layer, which is composed of large caliber, non-fenestrated vessels, are targeted. In another embodiment, hemichannels comprising vessel and endothelial cell connexins in cells of the inner choroid, also known as Sattler's layer, which is composed of significantly smaller vessels, are targeted. In another embodiment, hemichannels comprising connexins in cells of the outer and inner choroid are targeted. In another embodiment, hemichannels comprising connexins in capillaries of the choriocapillaris are targeted. In one embodiment, hemichannel vessel connexins targeted in methods of the invention include hemichannel connexins in pericytes and connexins in vascular smooth muscle and endothelial cells. In another embodiment, hemichannel vessel connexins targeted in methods of the invention include hemichannels in pericytes and connexins in endothelial cells, for example, in the microcapillaries. Cx43 hemichannels are a preferred target of the invention.

Another embodiment of this aspect of the invention provides a pharmaceutical pack that includes a small molecule or other hemichannel blocker. In one embodiment, the hemichannel blocker is Xiflam (tonabersat).

In another embodiment, the hemichannel blocker comprises, consists essentially of, or consists of Peptide5, GAP9, GAP19, GAP26, GAP27 or α-connexin carboxy-terminal (ACT) peptides, e.g., ACT-1 or other active anti-hemichannel peptidomimetics.

The activity of hemichannel blockers may be evaluated using certain biological assays. Effects of known or candidate hemichannel blockers on molecular motility can be identified, evaluated, or screened for using the methods described in the Examples below, or other art-known or equivalent methods for determining the passage of compounds through connexin hemichannels. Various methods are known in the art, including dye transfer experiments, for example, transfer of molecules labelled with a detectable marker, as well as the transmembrane passage of small fluorescent permeability tracers, which has been widely used to study the functional state of hemichannels. Various embodiments of this aspect of the invention are described herein, including a method for use in identifying or evaluating the ability of a compound to block hemichannels, which comprises: (a) bringing together a test sample and a test system, said test sample comprising one or more test compounds, and said test system comprising a system for evaluating hemichannel block, said system being characterized in that it exhibits, for example, elevated transfer of a dye or labelled metabolite, for example, in response to the introduction of hypoxia or ischemia to said system, a mediator of inflammation, or other compound or event that induces hemichannel opening, such as a drop in extracellular Ca²⁺; and, (b) determining the presence or amount of a rise in, for example, the dye or other labelled metabolite(s) in said system. Positive and/or negative controls may be used as well. Optionally, a predetermined amount of hemichannel blocker (e.g., Xiflam) may be added to the test system. Other methods useful to evaluate hemichannel blocker activity include electrophysiology and channel conductance block techniques, reduction in cytoplasmic swelling or cell edema, and reduced potassium efflux from cells, all of which are known in the art.

In one aspect, methods are provided for confirming, measuring or evaluating the activity of compounds useful for restoring or rescuing retinal function using assays, including tests using ARPE-19 cells. See Dunn K C, et al., ARPE-19, a human retinal pigment epithelial cell line with differentiated properties. Exp Eye Res. 1996 February; 62(2):155-69. Art methods may be used for confirming, measuring or evaluating the activity of compounds useful for restoring or rescuing choroidal structure and function. For example, choroidal thickness can be measured using ultrasonography, magnetic resonance imaging (MRI), and enhanced depth imaging optical coherence tomography (EDI-OCT). EDI-OCT is a noninvasive modality that enables cross-sectional imaging of the retina and choroid and has been used to measure choroidal thickness with acceptable reproducibility and sensitivity. Choroidal thickness has shown a positive correlation with retinal function, with a thicker choroid related to a better retinal function as measured, for example, with multifocal electroretinaogram (mfERG). Other retina-choroidal anatomy evaluation methods may be used for confirming, measuring or evaluating the activity of compounds useful for restoring or rescuing choroidal function, including swept-source OCT (SS-OCT).

### BRIEF DESCRIPTION OF THE FIGURES

FIG. 1 shows raw ECG waveforms for vehicle or drug-treated animals (A); the effects of vehicle and a hemichannel modulator (tonabersat) on mixed a-wave and b-wave amplitude of the ERG using 0.26 mg/kg (B&E), 0.8 mg/kg (C&F) and 2.4 mg/kg (D&G) tonabersat. Vehicle data shown is at 2 weeks post-injury; in these animals there is no recovery of ERG function. Statistical analysis was performed using a two-way ANOVA and a Bonferroni post-hoc test. Significant values are indicated with asterisks: *p<0.05; **p<0.01; ***p<0.001.

FIG. 2 shows the effect of vehicle and 2.4 mg/kg of a hemichannel modulator (tonabersat) on mixed a-wave (A) and b-wave (B) amplitude on the ERG 3 months post treatment of light damaged rats. The rod PIII (C) and PII (D) analysis shows untreated animals have significantly reduced amplitudes compared to their amplitude prior to light damage. Treated animals have maintained retinal function, matching the controls for Rod PII and only slightly lower for Rod PIII. All average data are expressed as mean±SEM. Statistical analysis was performed using a two-way ANOVA and a Bonferroni post-hoc test of the a- and b-wave values. Statistical analysis of rod PII and PIII was performed using an unpaired t-test with a Welch's correction. Significant values are indicated with asterisks: ***p<0.001. LD=Light Damage.

FIG. 3 shows the effect of oral delivery of a hemichannel modulator (tonabersat) on the light-damaged rat retinal and choroidal thickness. Fundus images and optical coherence tomography (OCT) images of normal Sprague Dawley (SD) rats (A), 2.4 mg/kg tonabersat treated animals 2 weeks after light damage (B) and vehicle treated light damaged rat (C). The green line on the fundus image represents the scan location of the adjacent cross-sectional OCT image. The coloured lines on the OCT images highlight the inner limiting membrane (cyanin), the OPL (orange), the ONL (orange to yellow), the choroid (green to red). Quantification shows that both the ONL and choroid are thinner by 2 weeks post light damage in vehicle treated animals compared to normal (pre-light exposure). Treated animals for each of the three tonabersat doses used did not show thinning in the ONL or choroid at any of the time points analyzed, 24 hours, 1 week and 2 weeks post light damage (rows D-F). Whilst there was some thinning at the lowest and middle dose, it was not significant. **=p<0.01; ***=p<0.001. Scale bar=100 μm

FIG. 4 shows the effect of vehicle (A) or treatment with a hemichannel modulator (tonabersat, 2.4 mg/kg) 3 months after light damage (B). Representative OCT images show significant thinning in the vehicle treated animals, with thinning evident especially in the INL, ONL and choroid. The colored lines on the OCT images highlight the inner limiting membrane (cyanin), the INL (orange to yellow), the ONL (yellow to red), the choroid (red to purple) and the sclera (purple to green). Measurements of the INL, ONL and choroid thickness are shown in C-E for retina prior to injury, vehicle treated at 3 months post LD, and tonabersat treated 3 months post LD. Data are expressed as mean±SEM. Significant values in comparison with light-damaged vehicle (peanut butter) group are indicated with asterisks: *p<0.05; **p<0.01; ***p<0.001. LD=Light Damage. Scale bar=100 μm

FIG. 5 shows an immunohistochemical analysis of the effects of three concentrations of an orally delivered hemichannel modulator (tonabersat) on light damaged rats. Orally treated rats showed less connexin43 immunoreactivity in the retina for all three dose levels (B-D) compared to vehicle group (A). Iba-1 immunolabelled cells showed low activation (sprouting) in the IPL of the retina of tonabersat-treated rats for all three doses (F-H) compared to vehicle-treated rats (E), although a slight increase in Iba-1 reactivity in the lowest 0.26 mg/kg oral dose was evident. GFAP immunoreactivity did not increase in the retina of 0.8 mg/ml (K) and 2.4 mg/kg (L) as compared to vehicle rats (I). In the lowest 0.26 mg/kg oral dose there was slightly increased GFAP labelling but it was still less expression than for the vehicle alone (J). Abbreviations: CGL: ganglion cell layer; IPL inner plexiform layer. Scale bar: 50 km.

FIG. 6 shows quantification of GFAP immunoreactive area (A), Connexin43 expression (B) and mean number of Iba-1 activated cells (C) in each of the three oral tonabersat dose level treated animals compared with vehicle alone of light-damaged rats. Analysis revealed significantly less upregulation of GFAP and Connexin43 in all three tonabersat treated groups compared with vehicle (p<0.001) (A-B). Quantification of the Iba-1 positive cells revealed a significantly reduced number of active microglia in all three tonabersat treated groups compared with vehicle (p<0.001) (C). Statistical analysis was conducted using one-way ANOVA, followed by Tukey's multiple comparisons test. Significant values in comparison with results from the untreated group are indicated with asterisks: ***p<0.001

FIG. 7 shows representative images of OCT of the hyperglycemic rats showing an average of 5-8 hyperreflective spots per eye (based upon 7 evenly spaced OCT scans across the retina and therefore an underestimate for the whole eye), but none in normal SD rats (A). The hyperreflective spots appeared to be microaneurysms (less than 20 μm diameter; arrows in BO and macroaneurysms (140-160 μm; arrow in C) and they were located specifically in the INS and ONL. The coloured lines on the OCT images highlight the INL (orange to yellow), the ONL (yellow to red), the choroid (purple to cyan). Evans Blue dye perfusion confirmed blood vessel leakage at sites of the aneurysms mapped using OCT. The green line on the fundus image (D) shows where the OCT scan (E) was taken. The hyperreflective spot (arrow) is a microaneurysm. The rats were injected with Evans Blue and the retina was then removed and imaged in that region revealing a region of vessel leak (F). Leakage was not associated with all microaneurysms but blood vessel leakage was consistently seen in four hyperglycemic rats with microaneurysms. Scale bar=100 μm

FIG. 8 shows an ERG analysis of hyperglycemic retina function 5 weeks after birth compared with normal SD rats from which the hyperglycemic strain was derived. Representative ERG mixed a- and b-waveforms are shown in A, B. The average mixed a-wave amplitude was significantly reduced in hyperglycemic rats compared to normal SD rats. Mixed b-wave amplitude was also significantly reduced in hyperglycemic rats, with normal SD rats. Breakdown analysis shows amplitudes were significantly reduced in the hyperglycemic rats for rod PIII (C), PII (D), cone PII (E) responses and for OPs control group. Statistical analysis was conducted using one-way ANOVA, followed by Tukey's multiple comparisons test. Significant values in comparison with normal SD are indicated with asterisks: **p<0.01; ***p<0.001. OP=oscillatory potentials.

FIG. 9 shows OCT and ERG analyses of hyperglycemic rat retinal structure and function at 8 weeks (at the lowest dose used, 0.28 mg/kg) once daily for 14 days (weeks 5-7) compared with vehicle treated animals. (A) shows a hyperreflective spot that is barely visible after treatment (B). ERG had significantly recovered in treated hyperglycemic rats compared to vehicle treated rats, whilst untreated rats had deteriorated further from week 5 to 8. In treated animals mixed a-wave was significantly higher compared to vehicle treated animals at 8 week (C). Similarly, mixed b-wave had significantly recovered in the tonabersat treated animals for all intensities, compared to vehicle control group (D). Further analysis revealed that treated hyperglycemic rats had significantly recovered rod PIII (E), PII (F), cone PII (G) and summed OPs (H) amplitudes. Statistical analysis was conducted using one-way ANOVA, followed by Tukey's multiple comparisons test. Significant values in comparison with vehicle treatment are indicated with asterisks: **p<0.01; ***p<0.001. OP=oscillatory potentials. Scale bar=100 μm

FIG. 10 shows immunohistochemical labelling in tonabersat-treated and vehicle-treated hyperglycemic rats at 8 weeks of age. GFAP labelling was intense in the CGL, where astrocytes are resident in areas around microaneurysm in the hyperglycemic rat retina, extending from the nerve fibre layer to the ONL indicating Müller cell activation (A). There was abnormally high Iba-1 labelling in the hyperglycemic retina (B) in the IPL where cells with enlarged soma and numerous elongated branches were present and connexin43 labelling was abnormally high in the GCL of the untreated animals (C). Hyperglycemic rats that had been fed daily with tonabersat for 14 days had reduced inflammation as evident by labelling of all three markers (D-F). Quantification of the results in G-I show that all three markers, GFAP, connexin43 and Iba-1 were significantly higher in vehicle-treated rats compared to undamaged control retina and tonabersat treatment resulted in significantly reduced labelling at 8 weeks, and significantly less than untreated rat retina levels. Statistical analysis was conducted using one-way ANOVA, followed by Tukey's multiple comparisons test. Significant values in comparison with results from the untreated group are indicated with asterisks: ***p<0.001. Scale bars=100 μm.

### DETAILED DESCRIPTION

Increased connexin43 hemichannel opening is associated with inflammasome pathway activation and inflammation in a range of pathologies including ocular disorders. We have discovered the utility of clinically safe doses of connexin hemichannel blockers, such as orally-delivered small molecule connexin hemichannel blockers, including Xiflam, in the restoration and rescue of retinal function and morphology, as well as choroidal function and structure, using the light-damaged retina animal model of dry AMD and a spontaneous rat model of DR. Clinical parameters (fundus imaging, optical coherence tomography (OCT) and electroretinogram) and inflammatory markers (immunohistochemistry for Iba-1 microglial marker, astrocyte marker glial fibrillary acidic protein and connexin43 protein expression) were assessed and showed that hemichannel blocker treatment led to the preservation of retinal photoreceptor function when assessed up to 3 months post light damage in the dry AMD model. In the DR model, clinical signs, including the presence of aneurysms confirmed using Evans blue dye perfusion, were reduced after daily tonabersat treatment for two weeks. Inflammation was also reduced and retinal function restored. We have discovered that hemichannel blockers can be used to not only improve, but restore, anatomical and functional outcomes in chronic retinal diseases.

Surprisingly, it was discovered that a single dose of a hemichannel blocker, ingested orally, was neuroprotective over an assessed period assessed out to 3 months post-acute light damage injury. It was discovered that the hemichannel blocker treatment significantly preserved the function of the retina, in particular the function of photoreceptors and bipolar cells in the inner retina. Furthermore, the increased oscillatory potentials identified using each of the three hemichannel blocker doses studied is indicative of a effect to preserve inner retinal cells despite the light-damage. The improved PIII and PII responses in the electroretinogram (ERG) also demonstrate specific preservation of the phototransduction pathway and postphotoreceptor neuron response. This study, described in Example 2, also showed that hemichannel blockers can be used to preserve the retinal layer structure as measured by OCT. See Example 2 for further details of these discoveries.

Additionally, as shown in Example 3, it was discovered that hemichannel blockers, for example, the oral blocker, Xiflam, are effective in shutting down signs of DR occurring secondary to spontaneous and chronic systemic hyperglycemia in a diabetic SD rat model. Signs of micro- and macro-aneurysms in the retina accompanied by an impact on visual retinal function were discovered in this phenotypical model of diabetes and DR.

The use of an oral hemichannel blocker, in this case Xiflam, compared to placebo controls, demonstrated regression of micro- and macro-aneurysms and significant rescue of retinal function as measured by ERG. The results from these quite disparate models have implications for other chronic ocular inflammatory diseases, in particular those involving the inflammasome pathway. Hemichannel blockade in the macular degeneration and diabetic retinopathy models described in this patent not only reduced inflammation, but surprisingly spared and rescued retinal structure and function, as well as, and importantly, choroidal structure.

This application relates to the surprising discovery of the modulation of hemichannel opening which has direct and long-lasting effects on the maintenance and rescue of retinal structure and function, as well as choroidal structure. See Examples 1-3 below. These discoveries that have important implications in the treatment of various diseases, disorders and conditions characterized in whole or in part by loss of retinal structure and/or function, including in diabetic retinopathy, which has no known cure.

It has also been discovered that hemichannel blockers including, for example, connexin 43 hemichannel blockers, can be used to preserve the chloride. Thus, hemichannel blockers can be used for methods to preserve choroidal function in disease states.

### Definitions

As used here, the term “about” a value or parameter refers to its meaning as understood in the art and includes embodiments that are directed to that value or parameter per se. For example, description referring to “about X” includes description of “X.” For example, the term “about 5 mg” of a weight value in a dosage refers to +1-0.5 degrees of the weight value.

A “small molecule” is defined herein to have a molecular weight below about 600 to 900 daltons, and is generally an organic compound. A small molecule can be an active agent of a hemichannel blocker prodrug. In one embodiment, the small molecule is below 600 daltons. In another embodiment, the small molecule is below 900 daltons.

As used herein, “treatment” (and grammatical variations thereof such as “treat” or “treating”) refers to clinical intervention to alter the natural course of the individual, tissue or cell being treated, and can be performed either for prophylaxis or during clinical pathology. Desirable effects of treatment include, but are not limited to, preventing occurrence or recurrence of a disease, disorder or condition, alleviation of signs or symptoms, diminishment of any direct or indirect pathological consequences of the disease, decreasing the rate of disease progression, amelioration or palliation of the disease state, and remission or improved prognosis. In some embodiments, compounds, methods and compositions of the invention can be used to delay development of a disease, disorder or condition, or to slow the progression of a disease, disorder or condition. The term does not necessarily imply that a subject is treated until total recovery. Accordingly, “treatment” includes reducing, alleviating or ameliorating the symptoms or severity of a particular disease, disorder or condition or preventing or otherwise reducing the risk of developing a particular disease, disorder or condition. It may also include maintaining or promoting a complete or partial state of remission of a condition.

“Treatment” as used herein also includes preserving and/or rescuing retinal structure, preserving and/or rescuing retinal function, preserving and/or rescuing choroidal structure, and/or preserving and/or rescuing choroidal function in a subject, following administration of a hemichannel blocker. A preferred hemichannel blocker is Xiflam. A preferred route of the administration is oral.

The term “treating” a disease, condition or disorders or the like, may refer to preventing, slowing, reducing, decreasing and, notably, to stopping and reversing the disorder, disease or condition, and/or improving and rescuing or restoring or normalizing retinal structure and/or function, and/or improving and rescuing or restoring or normalizing choroidal structure and/or function. In particular, for example, in stopping or reversing a disorder, disease or condition, or rescuing retinal function and/or structure, or rescuing choroidal function and/or structure, one or more or all of the symptoms of the disorder, disease or condition are reversed or substantially eliminated, the ONL in the retina is rescued, restored, and/or normalized, retinal ERG function, inner retinal function, retinal photoreceptor function (particularly rod photoreceptor function), and/or retinal PIII and PI rod responses is/are rescued, restored, and/or normalized, and the choriocapillaris in the choroid is rescued, restored, and/or normalized, respectively.

In other embodiments, the outer and inner nuclear layer of the retina are protected using the compounds and methods described herein, as shown in the Examples, which is important in chronic retinal diseases, including age-related macular degeneration, where the protective effects of the invention also find utility.

The term “preventing” means preventing in whole or in part, or ameliorating, or controlling.

As used herein, “effective amount” refers to an amount effective, at dosages and for periods of time necessary, to achieve the desired therapeutic or prophylactic result. For example, and not by way of limitation, an “effective amount” can refer to an amount of a compound or composition, disclosed herein, that is able to treat the signs and/or symptoms of a disease, disorder or condition that involve impaired retinal and/or choroidal structure and/or function, or to an amount of a hemichannel compound or composition that is able to beneficially modulate and rescue impaired retinal and/or choroidal structure and/or function.

As used herein, “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is preferably also one in which any toxic or detrimental effects of the substance/molecule, agonist or antagonist may be outweighed by the therapeutically beneficial effects. A therapeutically effective amount of a hemichannel blocker will beneficially maintain or improve retinal structure and/or function, and/or maintain or improve choroidal structure and/or function, in a subject.

As used herein, “prophylactically effective amount” refers to an amount effective, at dosages and for periods of time necessary, to achieve a desired prophylactic result, typically maintenance of rescued or restored retinal and/or choroidal function and/or structure. Typically, but not necessarily, the prophylactically effective amount will be less than the therapeutically effective amount.

The term “pharmaceutical formulation” refers to a preparation which is in such form as to permit the biological activity of an active ingredient contained therein, e.g., a hemichannel blocker, to be effective, and which does not contain additional components that are unacceptably toxic to a subject to whom the formulation would be administered.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, buffers, excipients, stabilizers, and preservatives.

As used herein, the term “subject” or the like, including “individual,” and “patient”, all of which may be used interchangeably herein, refers to any mammal, including humans, domestic and farm animals, and zoo, wild animal park, sports, or pet animals, such as dogs, horses, cats, sheep, pigs, cows, etc. The preferred mammal is a human, including adults, children, and the elderly. Preferred sports animals are horses and dogs. Preferred pet animals are dogs and cats. In certain embodiments, the subject, individual or patient is a human.

As used herein, the term “hemichannel” is a part of a gap junction (two hemichannels or connexons connect across an intercellular space between adjacent cells to form a gap junction) and is comprised of a number of connexin proteins, typically homologous or heterologous, i.e., homo- or hetero-meric hexamers of connexin proteins, that form the pore for a gap junction between the cytoplasm of two adjacent cells. The hemichannel is supplied by a cell on one side of the junction, with two hemichannels from opposing cells normally coming together to form the complete intercellular hemichannel. However, in some cells, and in cells under some circumstances, the hemichannel itself is active as a conduit between the cytoplasm and the extracellular space allowing the transfer of ions and small molecules.

Compounds of Formula I, for example Xiflam, and/or an analogue or pro-drug of any of the foregoing compounds, can modulate the function and/or activity of hemichannels, preferably those comprising any type of connexin protein. Accordingly, reference to “hemichannel” should be taken broadly to include a hemichannel comprising, consisting essentially of, or consisting of any one or more of a number of different connexin proteins, unless the context requires otherwise. However, by way of example, a hemichannel may comprise one or more of any connexin, including those referred to specifically above. In one embodiment, a hemichannel consists of one of the aforementioned connexins. In one embodiment, a hemichannel comprises one or more of connexin 36, 37, 40, 43, 45 and 57. In one embodiment, a hemichannel consists of one of connexin 37, 40, or 43. In one embodiment, the hemichannel is a connexin 43 hemichannel. In one embodiment, a hemichannel is retinal hemichannel. In one embodiment, hemichannel is choroidal hemichannel. In one embodiment, the a vascular hemichannel. In one embodiment, a hemichannel is a connexin hemichannel found in vascular endothelial cells. In one particular embodiment, a hemichannel comprises one or more of connexin 30, 37 and connexin 43. In one particular embodiment, a hemichannel consists of connexin 30. In one particular embodiment, a hemichannel consists of connexin 37. In one particular embodiment, a hemichannel consists of connexin 43. In one embodiment, the hemichannel comprises one or more connexins excluding connexin 26. In one embodiment, the composition can include or exclude a hemichannel blocker of any connexin, including the foregoing.

Hemichannels and hemichannels may be present in cells of any type. Accordingly, reference to a “hemichannel” or a “hemichannel” should be taken to include reference to a hemichannel or hemichannel present in any cell type, unless the context requires otherwise. In one embodiment of the invention, the hemichannel or hemichannel is present in a cell in an organ, or in a cancer or tumor. In one embodiment, the hemichannel is a vascular hemichannel. In one embodiment, the hemichannel is a connexin hemichannel found in vascular endothelial cells and/or vascular smooth muscle cells, or in the retinal and/or choroid or choroidal vasculature.

As used herein, “modulation of a hemichannel” is the modulation of one or more functions and/or activities of a hemichannel, typically, the flow of molecules between cells through a hemichannel. Such functions and activities include, for example, the flow of molecules from the extracellular space or environment through a hemichannel into a cell, and/or the flow of molecules through a hemichannel from the intracellular space or environment of a cell into the extracellular space or environment. Compounds useful for modulation of a hemichannel may be referred to as “hemichannel modulators.” All aspects of the inventions and methods described herein may be accomplished by modulation of a hemichannel.

Modulation of the function of a hemichannel may occur by any means. However, by way of example only, modulation may occur by one or more of: inducing or promoting closure of a hemichannel; preventing, blocking, inhibiting or decreasing hemichannel opening; triggering, inducing or promoting cellular internalization of a hemichannel and/or gap junction. Use of the words such as “blocking”, “inhibiting”, “preventing”, “decreasing” and “antagonizing”, and the like, may not be taken to imply complete blocking, inhibition, prevention, or antagonism, although this may be preferred, and shall be taken to include partial blocking, inhibition, prevention or antagonism to at least reduce the function or activity of a hemichannel and/or hemichannel. Similarly, “inducing” or “promoting” should not be taken to imply complete internalization of a hemichannel (or group of hemichannels) and should be taken to include partial internalization to at least reduce the function or activity of a hemichannel.

As used herein, the terms “anti-hemichannel compound” and “hemichannel blocker” is a compound that interferes with the passage of molecules through a connexin hemichannel. An anti-hemichannel compound or hemichannel blocker can block or decrease hemichannel opening, block or reduce the release of molecules through a hemichannel to an extracellular space, and/or block or reduce the entry of molecules through a hemichannel into an intracellular space. Anti-hemichannel compound and hemichannel blockers include compounds that fully or partially block hemichannel leak or the passage of molecules to or from the extracellular space. Anti-hemichannel compound and hemichannel blockers also include compounds that decrease the open probability of a hemichannel. Open probability is a measure of the percentage of time a channel remains open versus being closed (reviewed in Goldberg G S, et al., Selective permeability of gap junction channels Biochimica et Biophysica Acta 1662 (2004) 96-101). Anti-hemichannel compound and hemichannel blockers include hemichannel modulators. Anti-hemichannel compound and hemichannel blockers may interfere directly, or directly, with the passage of molecules through a connexin hemichannel. All aspects of the inventions and methods described herein may be accomplished by blocking a hemichannel, or decreasing the open probability of a hemichannel, for example, as described herein. In one embodiment, the connexin hemichannel is a connexin 43 hemichannel, and/or other vascular connexin hemichannel.

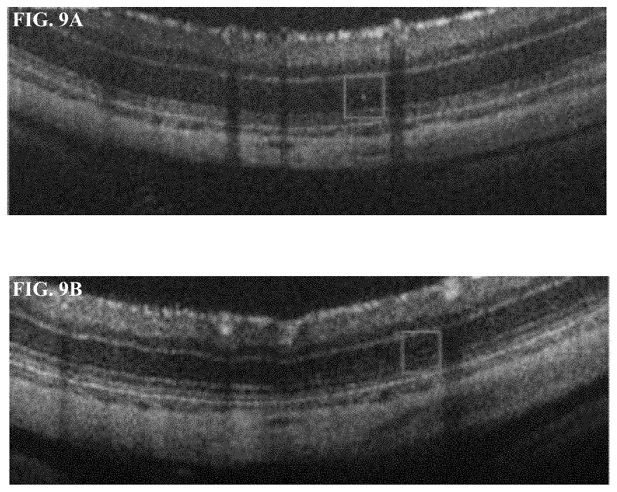

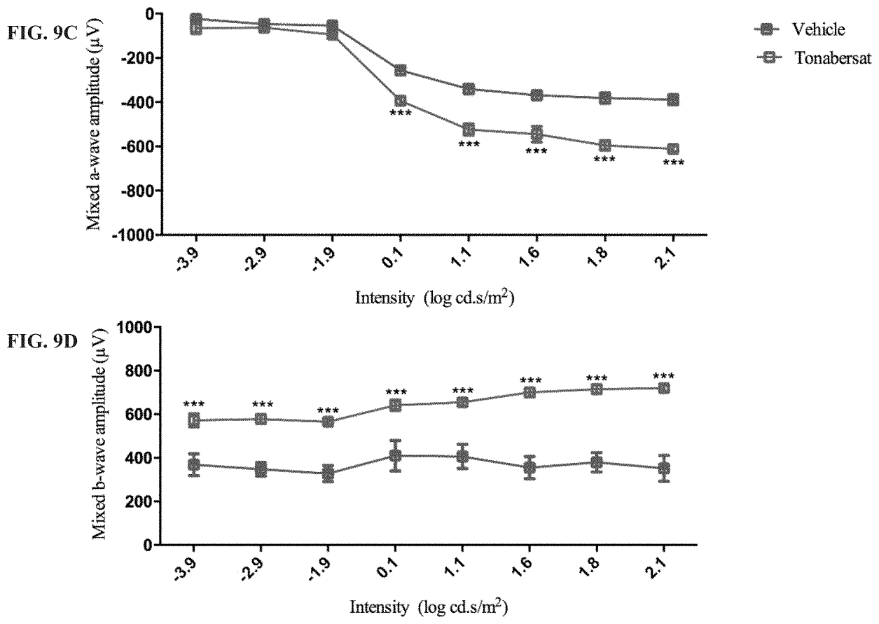

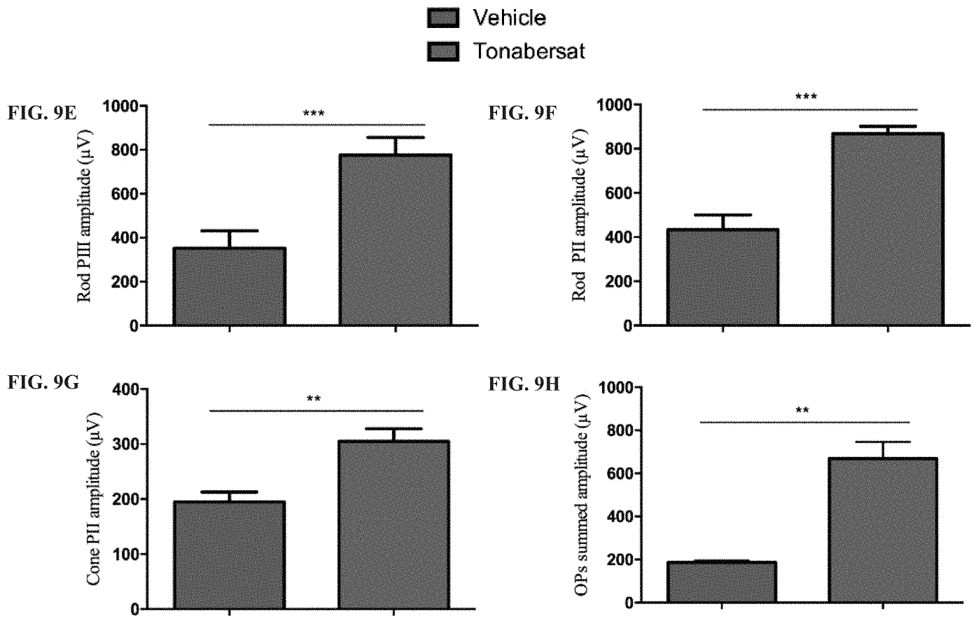

As used herein, the terms “restore or rescue retinal structure” and “rescue or restore retinal structure,” “rescuing and/or restoring retinal structure” and the like, refer to improving retinal structural integrity, including, for example, recovery of retinal pigment epithelium, recovery of retinal vascular endothelium, and/or recovery of normal retinal layer structure. The terms “restore or rescue retinal structure” et al. also refers to reducing or eliminating micro- and/or macro-aneurysms (see, e.g., FIG. 9B). In some embodiments of the invention, retinal structure is rescued and returned to a normal or pre-disease state. In some embodiments of the invention, retinal pigment epithelium, retinal vascular endothelium, and/or retinal layer structure are rescued and returned to a normal or pre-disease state.

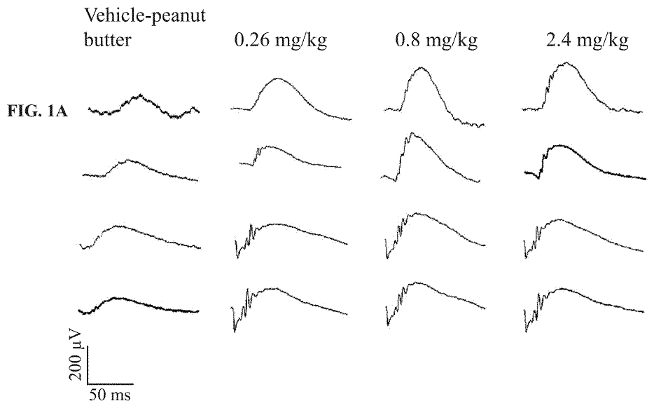

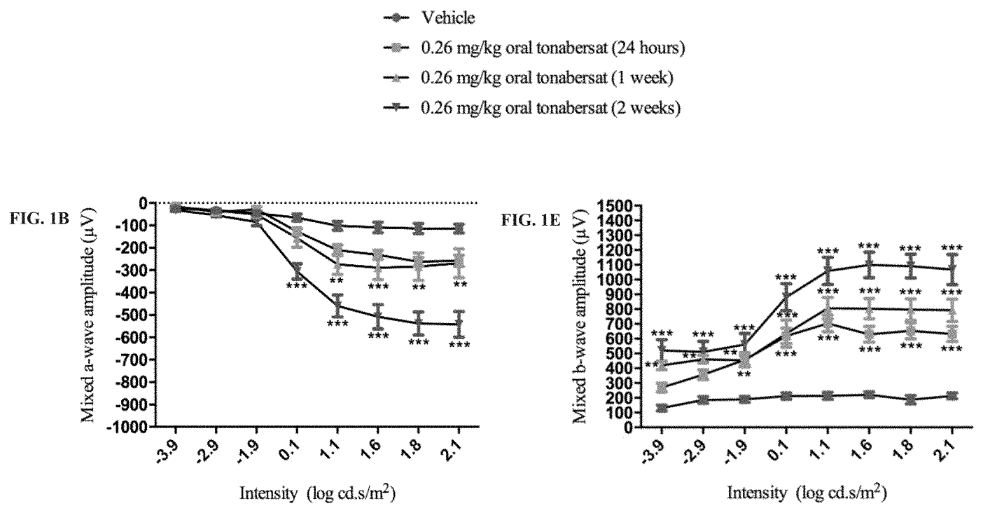

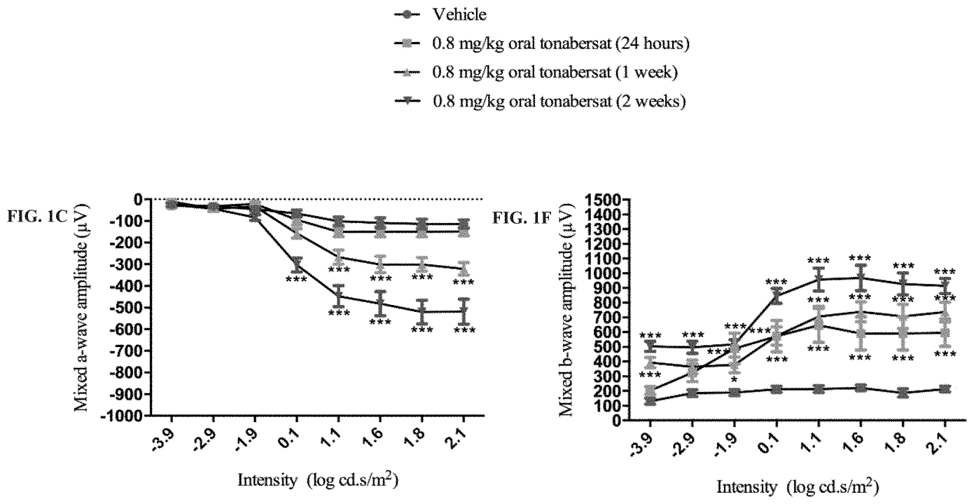

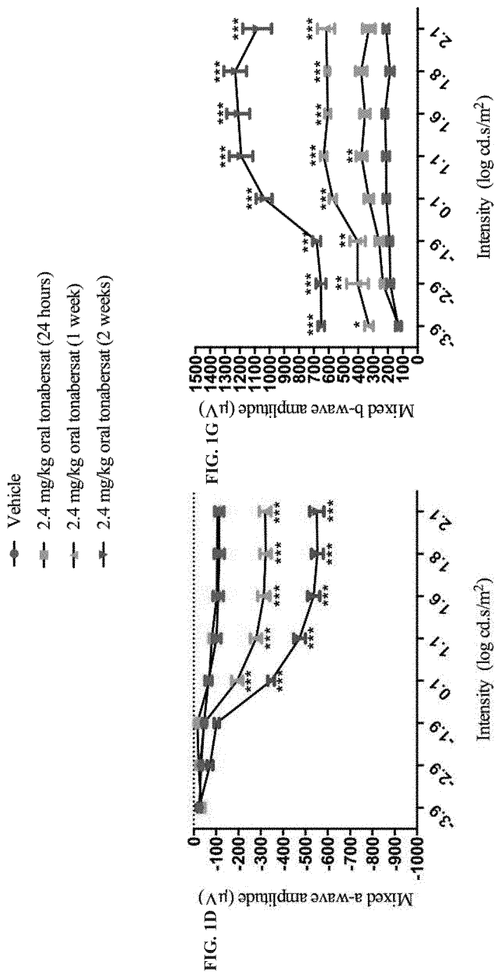

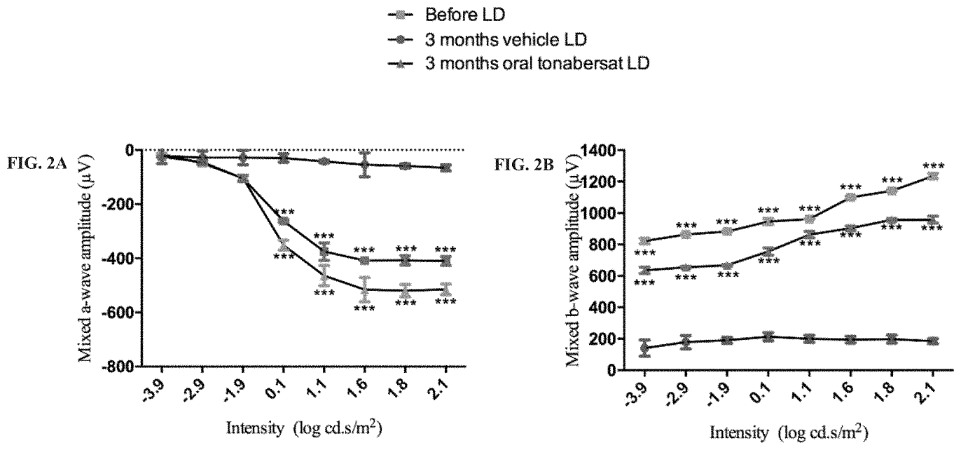

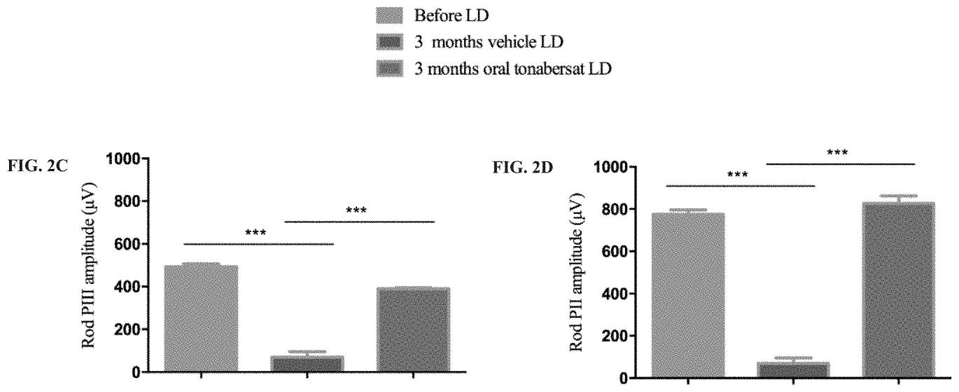

The terms “restore or rescue retinal function” and “rescue or restore retinal function,” “rescuing and/or restoring retinal function” and the like, refer to improving retinal function, including, for example, improving mixed a-wave function (see, e.g., FIG. 9C), improving mixed b-wave function (see, e.g., FIG. 9D) and/or improving PII and PIII rod and cone function (see, e.g., FIG. 9E-G), which may be evaluated, for example, by electroretinogram. The terms “restore or rescue retinal function” et al. also refer to improving overall ERG function. See also FIG. 1 which shows rescue of ERG function and inner retinal function, and FIG. 2 which shows improvement in photoreceptor function. In some embodiments of the invention, retinal function is rescued and returned to a normal or pre-disease state. In some embodiments of the invention, retinal ERG, PII and PIII rod and/or cone function, etc., are rescued and returned to a normal or pre-disease state.

As used herein, the terms “restore or rescue choroidal structure” and “rescue or restore choroidal structure,” “rescuing and/or restoring choroidal structure” and the like, refer to improving choroidal structural integrity, including, for example, recovery of choroidal thickness and/or recovery of the choroidal vascular bed, which may be determined, for example, using OCT angiography or fluorescin angiography. In some embodiments of the invention, choroidal structure is rescued and returned to a normal or pre-disease state. In some embodiments of the invention, choroidal thickness and/or the choroidal vascular bed are rescued and returned to a normal or pre-disease state.

As used herein, the terms “restore or rescue choroidal function” and “rescue or restore choroidal function,” “rescuing and/or restoring choroidal function” and the like, refers to improving choroidal blood flow, for example, which may be determined, for example, using high-speed OCT angiography. The terms “restore or rescue choroidal function” et al. also refers to improving the choroidal vascular blood flow to the outer retina, and improved modulation of choroidal blood flow. In some embodiments of the invention, choroidal function is rescued and returned to a normal or pre-disease state. In some embodiments of the invention, choroidal blood flow is rescued and returned to a normal or pre-disease state.

Compounds of the invention may be used in methods of treatment to preserve or rescue retinal structure, retinal function, choroidal structure and/or choroidal function, including in methods of treatment of diseases, disorders or conditions characterized in whole or in part by pathological, abnormal or otherwise unwanted or undesired diminution of retinal and/or choroidal structural or functional integrity. Integrity of the retina and/or chloride are essential to prevent loss of vision.

The terms “peptide,” “peptidomimetic” and “mimetic” include synthetic or genetically engineered chemical compounds that may have substantially the same structural and functional characteristics of protein regions which they mimic. In the case of connexin hemichannels, these may mimic, for example, the extracellular loops of hemichannel connexins.

The patent describes new methods to preserve or rescue retinal structure, retinal function, choroidal structure and/or choroidal function, which can be improved by the methods of the invention in a number of diseases, disorders or conditions, some of which are characterized by chronic retinal dysfunction and/or loss of retinal structure, and/or chronic choroid dysfunction and/or loss of choroidal structure.

The instant inventions provide, inter alia, methods for preservation or rescue of retinal structure, retinal function, choroidal structure and/or choroidal function by administration of a hemichannel blocker, such as compounds of Formula I, for example Xiflam, or compounds of Formula II, and/or an analogue or pro-drug of any of the foregoing compounds, for the treatment of a disease, disorder or condition characterized in whole or in part by loss of retinal structure, retinal function, choroidal structure and/or choroidal function.

In some embodiments, this invention features the use of compounds of Formula I, for example Xiflam, or compounds of Formula II, and/or an analogue or pro-drug of any of the foregoing compounds to directly and immediately block Cx43 hemichannels and to cause the preservation or rescue of retinal structure, retinal function, choroidal structure and/or choroidal function. Some exemplary doses are in the range of about 0.1 to about 5.0 mg/kg, including, for example, from 0.2 to 3.0 mg/kg, or from 0.2 to 2 mg/kg and from 0.2 to 1.0 mg/kg, or 0.2 to 0.5 mg/kg. Some exemplary daily or other periodic dose amounts range from about 10-250 mg per dose, including, for example, from about 20-25 mg per dose, from about 25-50 mg per dose from about 50-75 mg per dose, from about 75-100 mg per dose and from about 100-250 mg per dose, including doses of 20, 50, 100, and 150 mg per dose.

### Connexins

In various embodiments, the hemichannel being modulated is any connexin hemichannel, and may include or exclude a connexin 26 (Cx26) hemichannel. In certain embodiments, the hemichannel being modulated is a connexin 36 (Cx36) hemichannel, a connexin 37 (Cx37) hemichannel, a connexin 40 (Cx40) hemichannel, a connexin 43 (Cx43) hemichannel, a connexin 45 (Cx45) hemichannel, and/or a connexin 57 (Cx57) hemichannel. In one embodiment, the hemichannel being modulated comprises one or more of a Cx36, Cx37, Cx40, Cx43, Cx45 and/or Cx57 protein. In one particular embodiment, the hemichannel and/or hemichannel being modulated is a Cx37 and/or Cx40 and/or Cx43 hemichannel. In one particular embodiment, the hemichannel and/or hemichannel being modulated is a Cx30 and/or Cx43 and/or Cx45 hemichannel. In one particular embodiment, the hemichannel and/or hemichannel being modulated is a Cx36, Cx37, Cx43 and/or Cx45 hemichannel.

In some embodiments, the hemichannel being modulated can include or exclude any of the foregoing connexin proteins. In some aspects, the hemichannel blocker is a blocker of a Cx43 hemichannel, a Cx40 hemichannel and/or a Cx45 hemichannel. In certain preferred embodiments, the hemichannel blocker is a connexin 43 hemichannel blocker. The pharmaceutical compositions of this invention for any of the uses featured herein may also comprise a hemichannel blocker that may inhibit or block any of the noted connexin hemichannels (including homologous and heterologous hemichannels). In some embodiments the hemichannel being modulated can include or exclude any of the foregoing connexin hemichannels, or can be a heteromeric hemichannel.

The hemichannel blocker used in any of the administration, co-administrations, compositions, kits or methods of treatment of this invention is a Cx43 hemichannel blocker, in one embodiment. Other embodiments include Cx45 hemichannel blockers, Cx30 hemichannel blockers, Cx37 hemichannel blockers, Cx40 hemichannel blockers, and blockers of one or another of the connexin hemichannel or a hemichannel comprising noted above or herein, or consisting essentially of, or consisting of any other connexins noted above or herein. Some embodiments may include or exclude any of the foregoing connexins or hemichannels, or others noted in this patent. In various embodiments, by way of example, the hemichannel being modulated comprises one or more of connexin 36, connexin 37, connexin 40, connexin 43, connexin 45, connexin 57, connexin 59 and/or connexin 62.

In one embodiment, particularly as it relates to the retina, the hemichannel being modulated comprises one or more of a Cx36, Cx37, Cx40, Cx43, Cx45 or Cx57 protein. Targeted hemichannel connexins include one or more of selected hemichannel connexins in blood vessels (e.g, Cx37, Cx40 or Cx43), as well as hemichannel connexins in astroglial cells (e.g., Cx43), amacrine cells (e.g., Cx36, Cx45), bipolar cells (e.g., Cx36, Cx45), the outer and inner plexiform layer, the ganglion cell layer (e.g., Cx36, Cx45), cone photoreceptors and retinal endothelial cells, and other retinal neurons, for example. In some embodiments, Cx36 and Cx43 hemichannels are targeted. In one particular embodiment, the hemichannel and/or hemichannel being modulated comprises Cx43. In one embodiment, hemichannels comprising connexins in the cells of the outer plexiform layer are targeted (e.g., Cx43), where methods of the invention can stop and reverse OPL thinning and rescue the OPL.

In other embodiments, particularly those relating to the choroid or blood vessels of the retina, the hemichannel being modulated may preferentially comprise one or more of a Cx37, Cx40 or Cx43 protein. In one particular embodiment, the hemichannel and/or hemichannel being modulated comprises Cx43. In one embodiment, hemichannels comprising vessel connexins in cells of the outer choroid, also known as Haller's layer, which is composed of large caliber, non-fenestrated vessels, are targeted. In another embodiment, hemichannels comprising vessel and endothelial cell connexins in cells of the inner choroid, also known as Sattler's layer, which is composed of significantly smaller vessels, are targeted. In another embodiment, hemichannels comprising connexins in cells of the outer and inner choroid are targeted. In another embodiment, hemichannels comprising connexins in capillaries of the choriocapillaris are targeted. In one embodiment, hemichannel vessel connexins targeted in methods of the invention include hemichannel connexins in pericytes and connexins in vascular smooth muscle and endothelial cells. In another embodiment, hemichannel vessel connexins targeted in methods of the invention include hemichannels in pericytes and connexins in endothelial cells, for example, in the microcapillaries. Cx43 hemichannels are a preferred target of the invention.

### Small Molecule Hemichannel Blockers

Examples of hemichannel blockers include small molecule hemichannel blockers, e.g., Xiflam (tonabersat). The structure of tonabersat (also shown in PubChem, DrugBank, and MedChemExpress) is:

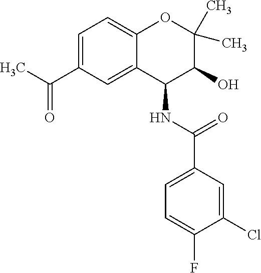 [SMILES: CC(=O)c1ccc2c(c1)[C@H](NC(=O)c1ccc(F)c(Cl)c1)[C@H](O)C(C)(C)O2]

Other chemical names for tonabersat are found in PubChem (N-[(3S,4S)-6-acetyl-3-hydroxy-2,2-dimethyl-3,4-dihydrochromen-4-yl]-3-chloro-4-fluorobenzamide), DrugBank (N-[(3S,4S)-6-acetyl-3-hydroxy-2,2-dimethyl-3,4-dihydro-2H-1-benzopyran-4-yl]-3-chloro-4-fluorobenzamide) and Chemical Book (N-((3S,4S)-6-Acetyl-3-hydroxy-2,2-dimethylchroman-4-yl)-3-chloro-4-fluorobenzamide; or 2H-Benzo(B)pyran-3-ol, 6-acetyl-4-(3-chloro-4-fluorobenzoylamino)-3,4-dihydro-2,2-dimethyl-; or N-[(3S,4S)-6-Acetyl-3,4-dihydro-3-hydroxy-2,2-dimethyl-2H-1-benzopyran-4-yl]-3-chloro-4-fluoro-benzamide).

In some embodiments, the hemichannel blocker is a small molecule other than Xiflam, for example, a hemichannel blocker described in Formula I or Formula II in US Pat. App. Publication No. 20160177298, filed in the name of Colin Green, et al., the disclosure of which is hereby incorporated in its entirety by this reference, as noted above. Various preferred embodiments include use of a small molecule that blocks or ameliorates or otherwise antagonizes or inhibits hemichannel opening, to treat the diseases, disorders and conditions described or referenced herein. In various embodiments, the small molecule that blocks or ameliorates or inhibits hemichannel opening is a prodrug of Xiflam or an analogue thereof.

In some embodiments, this invention features the use of small molecule hemichannel blockers including, for example, compounds of Formula I, such as Xiflam, and/or an analogue or pro-drug of any of the foregoing compounds to block Cx43 hemichannels, for example, for the rescue or restoration of retinal structure, rescue or restoration of retinal function, and for the rescue or restoration of choroidal structure and/or function.

By way of example, the hemichannel blocker Xiflam (tonabersat) may be known by the IUPAC name N-[(3S,4S)-6-acetyl-3-hydroxy-2,2-dimethyl-3,4-dihydrochromen-4-yl]-3-chloro-4-fluorobenzamide or (3S-cis)-N-(6-acetyl-3,4-dihydro-3-hydroxy-2,2-(dimethyl-d6)-2H-1-benzopyran-4-yl)-3-chloro-4-fluorobenzamide.

Another useful compound is boldine, an alkaloid of the aporphine class found in the boldo tree and in Lindera aggregata.

In one embodiment, Xiflam and/or an analogue or prodrug thereof is chosen from the group of compounds having the Formula I:

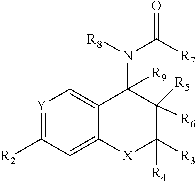

wherein,

### Y is C—R₁;

R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂—, CH(OH), SO₂, SO, CH₂—O—, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C₁₋₆ alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl.

In some embodiments, this invention features the use of small molecule hemichannel blockers including, for example, compounds of Formula II, and/or an analogue or pro-drug of any of the foregoing compounds to block Cx43 hemichannels, for example, for the rescue or restoration of retinal structure, rescue or restoration of retinal function, and for the rescue or restoration of choroidal structure and/or function.

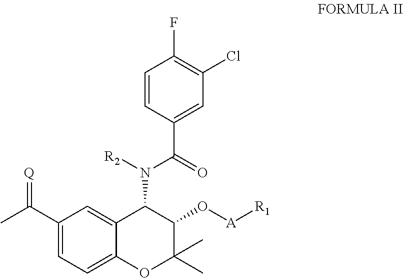

wherein
Q is O or an oxime of formula ═NHOR₄₃, wherein R₄₃ is (i) selected from H, C₁₋₄ fluoroalkyl or optionally substituted C₁₋₄ alkyl, or (ii) -A₃₀₀-R₃₀₀, wherein A₃₀₀ is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₃₀₀, R₃ and R₄ are selected independently from H, fluoro, C₁₋₄ alkyl, or C₁₋₄ fluoroalkyl, or R₃ and R₄ together with the atom to which they are attached form a cyclopropyl group, and R₃₀₀ is selected from groups [1], [2], [2A], [3], [4], [5] or [6];
R₂ is H,
A is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₁, R₃ and R₄ are selected independently from H, fluoro, C₁₋₄ alkyl, or C₁₋₄ fluoroalkyl, or R₃ and R₄ together with the atom to which they are attached form a cyclopropyl group,
R₁ is selected from groups [1], [2], [2A], [3], [4], [5] and [6] wherein the atom marked ** is directly connected to A:

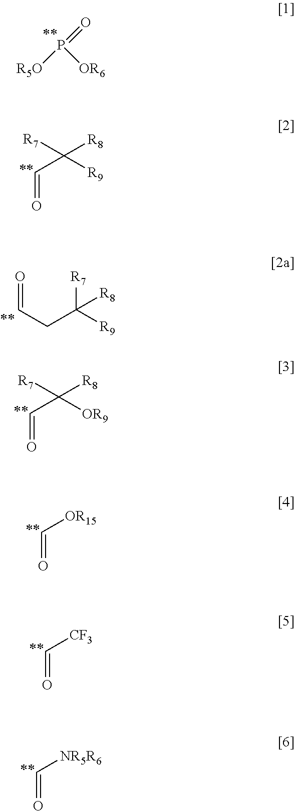

R₅ and R₆ are each independently selected from H, C₁₋₄ alkyl, C₁₋₄ fluoroalkyl, and benzyl;
R₇ is independently selected from H, C₁₋₄ alkyl, and C₁₋₄ fluoroalkyl;
R₈ is selected from:
(i) H, C₁₋₄ alkyl, or C₁₋₄ fluoroalkyl, or
(ii) the side chain of a natural or unnatural alpha-amino acid, or a peptidomimetic or other peptide as described herein, or
(iii) biotin or chemically linked to biotin;
R₉ is selected from H, —N(R₁₁)(R₁₂), or —N⁺(R₁₁)(R₁₂)(R₁₃)X⁻, or —N(R₁₁)C(O)R₁₄ wherein R₁₁, R₁₂, and R₁₃ are independently selected from H, C₁₋₄ alkyl, or C₁₋₄ fluoroalkyl,
R₁₄ is H, C₁₋₄ alkyl, or C₁₋₄ fluoroalkyl,
R₁₅ is independently selected from C₁₋₄ alkyl and C₁₋₄ fluoroalkyl, and
X⁻ is a pharmaceutically acceptable anion.

In some embodiments, Q is O.

For any of the Markush groups set forth above, that group can include or exclude any of the species listed for that group. Hemichannel blockers for use in methods of the invention may include or exclude any of these compounds.

In another embodiment, the analogue of Formula I is the compound carabersat (N-[(3R,4S)-6-acetyl-3-hydroxy-2,2-dimethyl-3,4-dihydrochromen-4-yl]-4-fluorobenzamide) or trans-(+)-6-acetyl-4-(S)-(4-fluorobenzoylamino)-3,4-dihydro-2,2-dimethyl-2H-1-benzo[b]pyran-3R-ol,hemihydrate.

In certain embodiments, Xiflam and/or an analogue thereof are in the form of a free base or a pharmaceutically acceptable salt. In other embodiments, one or more polymorph, one or more isomer, and/or one or more solvate of Xiflam and/or an analogue thereof may be used.

Other various small molecules have been reported to useful in inhibiting hemichannel activity. See Green et al., US Pat. App. Publication No. 20160177298, Formula II; Savory, et al., US Pat. App. Publication No. 20160318891; and Savory, et al., US Pat. App. Publication No. 20160318892, all of which are incorporated in their entireties by reference, as noted above. The hemichannel blockers for use in methods of the invention may include or exclude any of these compounds.

In one aspect, the invention relates to the use of pharmaceutical compositions, alone or within kits, packages or other articles of manufacture, in methods for treating diseases, disorders, or conditions noted herein, as well as those characterized by decreased or disordered retinal structure, retinal function, and/or choroidal structure. In some aspects, the hemichannel blocker is a connexin 43 hemichannel blocker. Blockers of other connexin hemichannels are within the invention, as noted.

In some embodiments “promoiety” refers to a species acting as a protecting group which masks a functional group within an active agent, thereby converting the active agent into a pro-drug. Typically, the promoiety will be attached to the drug via bond(s) that are cleaved by enzymatic or non-enzymatic means in vivo, thereby converting the pro-drug into its active form. In some embodiments the promoiety may also be an active agent. In some embodiments the promoiety may be bound to a hemichannel blocker molecule, peptide, antibody or antibody fragment. In some embodiments the promoiety may be bound to any of a peptide or peptidomimetic or small molecule or other organic hemichannel blocker, for example. In some embodiments the promoiety may be bound to a compound of Formula I. In some embodiments the pro-drug may be another hemichannel compound, e.g., a compound described in Green et al., US Pat. App. Publication No. 20160177298; Savory, et al., US Pat. App. Publication No. 20160318891; or Savory, et al., US Pat. App. Publication No. 20160318892.

### Chemical Delivery Modification

Hemichannel blockers useful in the present invention can also be formulated into microparticle (microspheres, Mps) or nanoparticle (nanospheres, Nps) formulations, or both, as well as liposomes or implants. Particulate drug delivery systems include nanoparticles (1 to 999 nm) and microparticles (1 to 1,000 μm), which are further categorized as nanospheres and microspheres and nanocapsules and microcaps. In nanocapsules and microcapsules, the drug particles or droplets are entrapped in a polymeric membrane. Particulate systems have the advantage of delivery by injection, and their size and polymer composition influence markedly their biological behavior in vivo. Microspheres can remain in the vitreous for much longer periods of time than nanospheres, therefore, microparticles act like a reservoir after injection. Nanoparticles diffuse rapidly and are internalized in tissues and cells.

Assessing Hemichannel Blocker Activity Various methods may be used for assessing the activity or efficacy of hemichannel blockers. In one aspect of the invention, the effects of hemichannel blocker treatment in a subject is evaluated or monitored using techniques to evaluate retinal structure, retinal function, and choroidal structure and/or function, as described herein, by way of example.

The activity of hemichannel blockers may also be evaluated using certain biological assays. Effects of known or candidate hemichannel blockers on molecular motility can be identified, evaluated, or screened for using the methods described in the Examples below, or other art-known or equivalent methods for determining the passage of compounds through connexin hemichannels. Various methods are known in the art, including dye transfer experiments, for example, transfer of molecules labelled with a detectable marker, as well as the transmembrane passage of small fluorescent permeability tracers, which has been widely used to study the functional state of hemichannels. See, for example, Schlaper, K A, et al. Currently Used Methods for Identification and Characterization of Hemichannels. Cell Communication and Adhesion 15:207-218 (2008). In vivo methods may also be used. See, for example, the methods of Danesh-Meyer, H V, et al. Connexin43 mimetic peptide reduces vascular leak and retinal ganglion cell death following retinal ischemia. Brain, 135:506-520 (2012); Davidson, J O, et al. (2012). Connexin hemichannel blockade improves outcomes in a model of fetal ischemia. Annals of Neurology 71:121-132 (2012).

One method for use in identifying or evaluating the ability of a compound to block hemichannels, comprises: (a) bringing together a test sample and a test system, said test sample comprising one or more test compounds, and said test system comprising a system for evaluating hemichannel block, said system being characterized in that it exhibits, for example, elevated transfer of a dye or labelled metabolite, for example, in response to the introduction of high glucose, hypoxia or ischemia to said system, a mediator of inflammation, or other compound or event that induces hemichannel opening, such as a drop in extracellular Ca²⁺; and, (b) determining the presence or amount of a rise in, for example, the dye or other labelled metabolite(s) in said system. Positive and/or negative controls may be used as well. Optionally, a predetermined amount of hemichannel blocker (e.g., Peptide5 or Xiflam) may be added to the test system.

### Dosage Forms and Formulations and Administration

All descriptions with respect to dosing, unless otherwise expressly stated, apply to the hemichannel blockers of the invention.

The hemichannel blockers can be dosed, administered or formulated as described herein.

In one embodiment, a composition comprising, consisting essentially of, or consisting of one or more hemichannel blockers are administered. Hemichannel blocker(s) may be administered QD, BID, TID, QID, or in weekly doses, e.g., QWK (once-per-week) or BIW (twice-per-week). They may also be administered monthly using doses described herein. They may also be administered PRN (i.e., as needed), and HS (hora somni, i.e., at bedtime).

The hemichannel blockers can be administered to a subject in need of treatment. Thus, in accordance with the invention, there are provided formulations by which a connexin hemichannel, for example, a connexin 43 hemichannel or a connexin 45 hemichannel or a connexin 36 hemichannel can be modulated to decrease its open probability in a transient and site-specific manner.

The hemichannel blockers may be present in the formulation in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 80%, 85%, or 90%, e.g. at least about 88%, at least about 90, 95 or 98%, or at least about 99% of a small molecule hemichannel blocker, for example, or dry mass of the preparation.

Administration of a hemichannel blocker to a subject may occur by any means capable of delivering the agents to a target site within the body of a subject. By way of example, a hemichannel blocker may be administered by one of the following routes: oral, topical, systemic (e.g., intravenous, intra-arterial, intra-peritoneal, transdermal, intranasal, or by suppository), parenteral (e.g. intramuscular, subcutaneous, or intravenous or intra-arterial injection), by implantation (including peritoneal, subcutaneous and ocular implantation), and by infusion through such devices as osmotic pumps, transdermal patches, and the like. Exemplary administration routes are also outlined in: Binghe, W. and B. Wang (2005). Drug delivery: principles and applications, Binghe Wang, Teruna Siahaan, Richard Soltero, Hoboken, N.J. Wiley-Interscience, c2005. In one embodiment, a hemichannel blocker is administered systemically. In another embodiment, a hemichannel blocker is administered orally. In another embodiment, a hemichannel blocker is administered topically onto or directly into the eye, for example.

In some aspects, the hemichannel blocker may be provided as, or in conjunction with, an implant. In some aspects, the implant may provide for slow-release, controlled-release or sustained-release delivery, with or without a burst dose. In some embodiments, a microneedle, needle, iontophoresis device or implant may be used for administration of the hemichannel blocker. The implant can be, for example, a dissolvable disk material such as that described in S. Pflugfelder et al., ACS Nano, 9 (2), pp 1749-1758 (2015). In some aspects, the hemichannel blockers, e.g. connexin 43 hemichannel blockers, of this invention may be administered via intraventricular, and/or intrathecal, and/or extradural, and/or subdural, and/or epidural routes.

The hemichannel blocker may be administered once, or more than once, or periodically. It may also be administered PRN (as needed) or on a predetermined schedule or both. In some aspects, the hemichannel blocker is administered daily, weekly, monthly, bi-monthly or quarterly, or in any combination of these time periods. For example, treatment may be administered daily for a period, follow by weekly and/or monthly, and so on. Other methods of administering blockers are featured herein. In one aspect, a hemichannel blocker is administered to a patient at times on or between days 1 to 5, 10, 30, 45, 60, 75, 90 or day 100 to 180, in amounts sufficient to treat the patient.

A hemichannel blocker, such as compounds of Formula I, for example Xiflam, and analogs or prodrugs of any of the foregoing compounds, or a compound of Formula II, may be administered alone or in combination with one or more additional ingredients and may be formulated into pharmaceutical compositions including one or more pharmaceutically acceptable excipients, diluents and/or carriers. In some embodiments, the hemichannel blocker, such as compounds of Formula I, for example Xiflam (tonabersat), and analogs or prodrugs of any of the foregoing compounds, or a compound of Formula II, may be orally administered in a composition comprising a foodstuff. In some embodiments, the foodstuff is peanut butter or a hazelnut-based cream. Without being bound by theory, it is believed that the relatively hydrophobic compounds of Formula I, including tonabersat, or Formula II, are slowly released after encapsulation in the emulsified fats of a foodstsuff (e.g., peanut butter), resulting in a prolonged therapeutic lifetime.

As used herein, the term “pharmaceutically acceptable diluents, carriers and/or excipients” is intended to include substances that are useful in preparing a pharmaceutical composition, may be co-administered with compounds of Formula I, for example Xiflam, and analogs of any of the foregoing compounds, or compounds of Formula II, while allowing it to perform its intended function, and are generally safe, non-toxic and neither biologically nor otherwise undesirable. Pharmaceutically acceptable diluents, carriers and/or excipients include those suitable for veterinary use as well as human pharmaceutical use. Suitable carriers and/or excipients will be readily appreciated by persons of ordinary skill in the art, having regard to the nature of compounds of Formula I, for example Xiflam, and analogs of any of the foregoing compounds. However, by way of example, diluents, carriers and/or excipients include solutions, solvents, dispersion media, delay agents, polymeric and lipidic agents, emulsions and the like. By way of further example, suitable liquid carriers, especially for injectable solutions, include water, aqueous saline solution, aqueous dextrose solution, and the like, with isotonic solutions being preferred for intravenous, intraspinal, and intracisternal administration and vehicles such as liposomes being also especially suitable for administration of agents.

Compositions may take the form of any standard known dosage form including tablets, pills, capsules, semisolids, powders, sustained release formulation, solutions, suspensions, elixirs, aerosols, liquids for injection, gels, creams, transdermal delivery devices (for example, a transdermal patch), inserts such as organ inserts, e.g., skin or eye, or any other appropriate compositions. Persons of ordinary skill in the art to which the invention relates will readily appreciate the most appropriate dosage form having regard to the nature of the condition to be treated and the active agent to be used without any undue experimentation. It should be appreciated that one or more of hemichannel blocker, such as compounds of Formula I, for example Xiflam, and analogs of any of the foregoing compounds, and/or a compound of Formula II, may be formulated into a single composition. In certain embodiments, preferred dosage forms include an injectable solution, an implant (preferably a slow-release, controlled-release or sustained-release implant, with or without a burst dose) and an oral formulation.

Compositions useful in the invention may contain any appropriate level of hemichannel blocker, such as compounds of Formula I, for example Xiflam, and analogs of any of the foregoing compounds, and/or a compound of Formula II, having regard to the dosage form and mode of administration. However, by way of example, compositions of use in the invention may contain from approximately 0.1% to approximately 99% by weight, preferably from approximately 1% to approximately 60% of a hemichannel blocker, depending on the method of administration.

In addition to standard diluents, carriers and/or excipients, a composition in accordance with the invention may be formulated with one or more additional constituents, or in such a manner, so as to enhance the activity or bioavailability of hemichannel blocker, such as compounds of Formula I, for example Xiflam, and analogs of any of the foregoing compounds, and/or a compound of Formula II, help protect the integrity or increase the half-life or shelf life thereof, enable slow release upon administration to a subject, or provide other desirable benefits, for example. For example, slow release vehicles include macromers, poly(ethylene glycol), hyaluronic acid, poly(vinylpyrrolidone), or a hydrogel. By way of further example, the compositions may also include preserving agents, solubilizing agents, stabilizing agents, wetting agents, emulsifying agents, sweetening agents, coloring agents, flavoring agents, coating agents, buffers and the like. Those of skill in the art to which the invention relates can identify further additives that may be desirable for a particular purpose.

As noted, hemichannel blockers may be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped compound. Liposomes containing hemichannel blockers may be prepared by known methods, including, for example, those described in: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mole percent cholesterol, the selected proportion being adjusted for the most efficacious therapy. Slow release delivery using PGLA nano- or microparticles, or in situ ion activated gelling systems may also be used, for example.

Additionally, it is contemplated that a hemichannel blocker pharmaceutical composition for use in accordance with the invention may be formulated with additional active ingredients or agents which may be of therapeutic or other benefit to a subject in particular instances. Persons of ordinary skill in the art to which the invention relates will appreciate suitable additional active ingredients having regard to the description of the invention herein and nature of the disorder to be treated.

Additionally, it is contemplated that a hemichannel blocker pharmaceutical composition for use in accordance with the invention may be formulated in a candy or food item, e.g, as a “gummy” pharmaceutical.

The compositions may be formulated in accordance with standard techniques as may be found in such standard references as Gennaro A R: Remington: The Science and Practice of Pharmacy, 20^{th} ed., Lippincott, Williams & Wilkins, 2000, for example. However, by way of further example, the information provided in US2013/0281524 or U.S. Pat. No. 5,948,811 may be used.

Any container suitable for storing and/or administering a pharmaceutical composition may be used for a hemichannel blocker product for use in a method of the invention.

The hemichannel blocker(s), for example, connexin 43 hemichannel blocker(s) may, in some aspects, be formulated to provide controlled and/or compartmentalized release to the site of administration. In some aspects of this invention, the formulations may be immediate, or extended or sustained release dosage forms. In some aspects, the dosage forms may comprise both an immediate release dosage form, in combination with an extended and/or sustained release dosage form. In some aspects both immediate and sustained and/or extended release of hemichannel blocker(s) can be obtained by combining hemichannel blocker(s) in an immediate release form. In some aspects of this invention the hemichannel blockers are, for example, connexin 43 blockers or other hemichannel blockers of this disclosure. In some aspects of this invention, the dosage forms may be implants, for example, biodegradable or nonbiodegradable implants.

The invention comprises methods for modulating the function of a hemichannel for the treatment and reversal or substantial reversal or amelioration of various disorders. Methods of the invention comprise administering a hemichannel blocker, alone or in a combination with one or more other agents or therapies as desired.

Administration of a hemichannel blocker, and optionally one or more other active agents, may occur at any time during the progression of a disorder, or prior to or after the development of a disorder or one or more symptom of a disorder. In one embodiment, a hemichannel blocker is administered periodically for an extended period to assist with ongoing management or reversal of symptoms. In another embodiment, a hemichannel blocker is administered periodically for an extended period or life-long to prevent or delay the development of or eliminate a disorder.

In some embodiments, the hemichannel blockers, for example, a connexin 43 hemichannel blocker (e.g., compounds of Formula (I), including tonabersat, or compounds of Formula (II)), can be administered as a pharmaceutical composition comprising one or a plurality of particles. In some aspects, the pharmaceutical composition may be, for example, an immediate release formulation or a controlled release formulation, for example, a delayed release particle. In other aspects, hemichannel blockers can be formulated in a particulate formulation one or a plurality of particles for selective delivery to a region to be treated. In some embodiments, the particle can be, for example, a nanoparticle, a nanosphere, a nanocapsule, a liposome, a polymeric micelle, or a dendrimer. In some embodiments, the particle can be a microparticle. The nanoparticle or microparticle can comprise a biodegradable polymer. In other embodiments, the hemichannel blocker is prepared or administered as an implant, or matrix, or is formulated to provide compartmentalized release to the site of administration. In some embodiments, the pharmaceutical composition of the hemichannel blockers, for example, a connexin 43 hemichannel blocker (e.g., compounds of Formula (I), including tonabersat, or compounds of Formula (II)) does not comprise microparticles.

In some embodiments, as noted, the formulated hemichannel blocker is a connexin 37 or connexin 40 or connexin 43 or connexin 45 hemichannel blocker, by way of example. Connexin 36 or connexin 37 or connexin 40 or connexin 43 or connexin 45 blockers are preferred. Most preferred are connexin 36 and connexin 43 hemichannel blockers. Especially preferred are connexin 43 hemichannel blockers. As used herein, “matrix” includes for example, matrices such as polymeric matrices, biodegradable or non-biodegradable matrices, and other carriers useful for making implants or applied structures for delivering the hemichannel blockers. Implants include reservoir implants and biodegradable matrix implants.

### Articles of Manufacture/Kits of Combinations of Connexin Hemichannel Blockers

In another embodiment of the invention, an article of manufacture, or “kit”, containing materials useful for treating the diseases and disorders described above is provided. The kit comprises a container comprising, consisting essentially of, or consisting of connexin hemichannel blocker. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The container holds a hemichannel blocker, or a formulation thereof, which is effective for treating the condition and may have a sterile access port (e.g., the container may be an intravenous solution bag or a vial having a stopper pierceable by a hypodermic injection needle). The label or package insert indicates that the composition is used for treating the condition of choice, such any of the diseases, disorders and/or conditions described or referenced herein. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the hemichannel blocker to a patient in need thereof.

Articles of manufacturer are also provided, comprising, consisting essentially of, or consisting of a vessel containing a hemichannel blocker compound, composition or formulation and instructions for use for the treatment of a subject. For example, in another aspect, the invention includes an article of manufacture comprising, consisting essentially of, or consisting of a vessel containing a therapeutically effective amount of one or more connexin hemichannel blockers, including small molecules, together with instructions for use, including use for the treatment of a subject.

In some aspects, the article of manufacture may comprise a matrix that comprises one or more connexin hemichannel blockers, such as a small molecule hemichannel blocker, alone or in combination.

### Doses, Amounts and Concentrations

As will be appreciated, the dose of hemichannel blocker administered, the period of administration, and the general administration regime may differ between subjects depending on such variables as the target site to which it is to be delivered, the severity of any symptoms of a subject to be treated, the type of disorder to be treated, size of unit dosage, the mode of administration chosen, and the age, sex and/or general health of a subject and other factors known to those of ordinary skill in the art.

Included herein are methods for increasing survival of, and rescuing or restoring, retinal structure and/or function and/or choroidal structure and/or function in a subject in need thereof, comprising, e.g., administering to said subject an effective amount of a hemichannel blocker, including, for example, N-[(3S,4S)-6-acetyl-3-hydroxy-2,2-dimethyl-3,4-dihydrochromen-4-yl]-3-chloro-4-fluorobenzamide (Xiflam). In some embodiments, the survival-promoting amount is about 10 to about 200 mg per day, or in some embodiments, from about 3.5 to 350 mg per day. In other embodiments, the survival-promoting amount is about 20 to about 100 mg per day. These amounts may be administered in single or divided doses, e.g., BID. Preferred are doses ranging from about 0.5 to about 5 mg/kg per day. Doses may be, for example, about 0.5, 0.6, 0.7, 0.8, 0.9, 1.0, 1.1., 1.2, 1.3, 1.4, 1.5 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4., 4.5, 4.6, 4.7, 4.8, 4.9, or about 5.0 mg/kg per day, or any range between any two of the recited doses.

An especially preferred daily dose is about 1.4 mg/kg per day, in single or divided doses (e.g., BID). Thus, for example, with a subject weighing about 70 kg, 90 kg, or 100 kg, the daily dose would be about 98 mg, about 126 mg or about 140 mg, respectively. These doses will provide an effective, peak steady state concentration of a hemichannel blocker, e.g., Xiflam, after about 10 days.

Importantly for efficacy and for patient convenience and compliance, other doses, as well as useful weekly, monthly and implant dosing and dosing regimens have also been discovered and are provided herein. Some preferred weekly doses range from about 2 mg/kg to about 50 mg/kg. The weekly dose may be for, example, about 2, 3, 4, 5, 6, 7, 8 9 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mg/kg, or any range between any two of the recited weekly doses. QWK dosing of from about 42 to about 47 mg/kg, for example, provide efficacious trough hemichannel concentrations for a hemichannel blocker with about 4-5 expected half-lifes per week, e.g., a hemichannel blocker of Formula I or II, for example, Xiflam, for carrying out methods of the invention with respect to retinal and/or choroidal structure and function. Plasma peak concentrations with doses from about 42 to about 47 mg/kg will be higher than efficacious trough concentrations but tolerated. Doses from 25-100 mg/kg will also be efficacious when administered monthly.

In some embodiments, the survival-promoting amount is about 4.5 to about 450 mg administered once per week (QWK). These doses include doses ranging from about 4.5 to about 45 mg QWK and from to about 45 to 450 mg per week (QWK), or any dose in between. Doses obtained by multiplying any of the weekly doses disclosed herein by the weight of a patient (e.g., 60, 65, 70, 75, 80, 85, 90, 95 or 100 kg) may also be used.

In another weekly QWK dosing embodiment, the hemichannel blocker compound is administered in a slow-release, sustained-release or controlled release oral or implant formulation, with or without a 10-20% burst dose, or other desired burst dose. Implant formulations, for example, ocular implant formulations, preferably range from disposed in a slow-release, sustained-release or controlled release oral or implant formulation

Dose amounts of of 3.5 to 350 mg per day, 10 to 200 mg per day or 20 to 100 mg per day, can also be used to rescue or restore retinal structure and/or function, and to rescue or restore choroidal structure and/or function. In some embodiments, oral doses of 15-150 mg, 25-250 mg, 40-400 mg or 80-800 mg of an anti-hemichannel compound is administered, in single or divided doses as an amount for promote the survival of retinal function and/or choroidal function, to rescue or restore retinal structure and/or function, or to to rescue or restore choroidal structure and/or function. In other embodiments, oral doses of 100-500 mg, 500-1000 mg, or 1000-2000 mg are administered, in single or divided doses. Divided doses are administered BID, TID or QID, or QWK. Xiflam is the presently preferred compound for oral dosing.

Importantly, weekly dosing will be useful to rescue or restore retinal structure and/or function, or to rescue or restore choroidal structure and/or function. Importantly, higher doses such as 500 mg to 2000 mg, or amounts in between these doses, for example, 750 mg, 1000 mg, 1250 mg, 1500 mg and 1750 mg, need only be administered once per week or even once per month for rescue or restoration of retinal structure and/or function, or for rescue or restoration of choroidal structure and/or function. Xiflam is the presently preferred compound for oral dosing in these amounts. Other QWK doses include doses from about 2500 to 5500 mg, with preferred doses equal to about 2900 mg, 3700 mg, 4200 mg, 3300 mg, 4200 mg and 4700 mg QWK, as well as all doses in between these. These doses are also efficacious when administered monthly.

Examples of effective doses that may be used for the treatment of the diseases, disorders or conditions referenced herein are described. Other exemplary doses are in the range of about 0.1 to about 5.0 mg/kg, including, for example, from 0.2 to 3.0 mg/kg, or from 0.2 to 2 mg/kg and from 0.2 to 1.0 mg/kg, or 0.2 to 0.5 mg/kg. Some exemplary daily or other periodic dose amounts range from about 10-250 mg per dose, including, for example, from about 20-25 mg per dose, from about 25-50 mg per dose, from about 20-40 mg per dose, from about 50-75 mg per dose, from about 75-100 mg per dose and from about 100-250 mg per dose, including doses of 20, 50, 100, and 150 mg per dose, or or any specific dose falling within one of these ranges of mg of drug per kg body weight. In some embodiments, the circulating concentration of the hemichannel blocker (including compounds of Formula (I), including tonabersat, and compounds of Formula (II)) in the subject to whom the hemichannel blocker was administered ranges from about 5 micromolar to about 200 micromolar, from about 7 micromolar to about 100 micromolar, or from about 10 micromolar to about 90 micromolar.

As noted above, doses of a hemichannel blocker, for example, a connexin 37, 40 or 43 hemichannel blocker, may be administered in single or divided applications. The doses may be administered once, or application may be repeated. Typically, application will be repeated weekly, biweekly, or every 3 weeks, every month, or every 2, 3, 4, 5, 6, 7, 8, 9, 10, 11, 12, 13, 14, 15, 16, 17, 18, 19, 20, 21, 22, 23, or every 24 months or more as needed to prevent, slow, or treat any disease, disorder or condition described herein. Doses may also be applied every 12 hours to 7 days apart, or more. For example, doses may be applied 12 hours, or 1, 2, 3, 4, 5, 6, or 7 days apart, or at any time interval falling between any two of these times, or between 12 hours and 7 days. The connexin 43 hemichannel blocker, for example, may be administered for up to four, six, eight, ten, twelve, fourteen, sixteen, eighteen, twenty, twenty-two, twenty-four or twenty-six weeks. For some indications, more frequent dosing, may employed. In some embodiments, the hemichannel blocker may be administered at a starting dosage level daily for a first period of time and then an increased dosage level daily for a further period of time.

### Manufacture and Purity

Small molecule hemichannel blockers, including those of Formula I and II may be prepared as previously described.

In some embodiments, the formulations of this invention are substantially pure. By substantially pure is meant that the formulations comprise less than about 10%, 5%, or 1%, and preferably less than about 0.1%, of any impurity. In some embodiments the total impurities, including metabolites of the connexin 43 modulating agent, will be not more than 1-15%. In some embodiments the total impurities, including metabolites of the connexin 43 modulating agent, will be not more than 2-12%. In some embodiments the total impurities, including metabolites of the connexin 43 modulating agent, will be not more than 3-11%. In other embodiments the total impurities, including metabolites of the connexin 43 modulating agent, will be not more than 4-10%.

### EXAMPLES

The work described in these Examples evaluated and demonstrated the ability of treatment with hemichannel blocker doses and dose regimens to maintain choroid thickness, to maintain retinal thickness, and to preserve and rescue retinal function in animals with diabetic retinopathy.

### Example 1

### Methods

Retina Light Damage Model of Dry Age-Related Macular Degeneration—The intense light damage model was prepared and carried out as described in previous studies. Mat Nor N, Guo C X, Rupenthal ID, Chen Y S, Green C R, Acosta M L. Sustained Connexin43 Mimetic Peptide Release From Loaded Nanoparticles Reduces Retinal and Choroidal Photodamage. Invest Ophthalmol Vis Sci. 2018; 59:3682-93; Guo C X, Mat Nor M N, Danesh-Meyer H V, Vessey K A, Fletcher E L, O'Carroll S J, et al. Connexin43 Mimetic Peptide Improves Retinal Function and Reduces Inflammation in a Light-Damaged Albino Rat Model. Invest Ophthalmol Vis Sci. 2016; 57:3961-73; Guo C X, Tran H, Green C R, Danesh-Meyer H V, Acosta M L. Gap junction proteins in the light-damaged albino rat. Mol Vis. 2014; 20:670-82; Noell W K, Walker V S, Kang B S, Berman S. Retinal damage by light in rats. Invest Ophthalmol. 1966; 5:450-73. The light damage rat model was selected as it allows direct comparison with this and other drugs. Kim Y, Griffin J M, Nor M N M, Zhang J, Freestone P S, Danesh-Meyer H V, et al. Tonabersat Prevents Inflammatory Damage in the Central Nervous System by Blocking Connexin43 Hemichannels. Neurotherapeutics. 2017; 14:1148-65; Mat Nor N, Guo C X, Rupenthal ID, Chen Y S, Green C R, Acosta M L, supra; Guo C X, Mat Nor M N, Danesh-Meyer H V, Vessey K A, Fletcher E L, O'Carroll S J, et al., supra. The model demonstrates pathological factors of AMD (oxidative stress and inflammation) and allows for measurable endpoints (including electrical function of the retina). The disadvantage of this model is that, like in other rodent models, drusen do not develop. All experimental procedures were approved by the University of Auckland Animal Ethics Committee, approval No. 001462 and comply with the Association for Research in Vision and Ophthalmology (ARVO) statement for the use of animals in eye research. Six to eight-week old albino Sprague Dawley (SD) rats (200-250 g; male or female) were used. Adult SD rats were exposed to continuous bright light for 24 hours, consistently starting at 9:00 am to minimize possible time-of-day variability. Light exposure was to two animals at a time to prevent rats from taking cover using each other as a shield. The LD protocol and intervention was repeated until the number of individuals for each dose group was obtained (n=7 per group). The light luminance was 2700 lux, generated using fluorescent lamps (Philips Master TLD 18W/965; Koninklijke Philips Electronics N.V., China) directly above the animal cages. The lamps were cool and emitted broadband light, from 380 to 760 nm wavelength, with the average intensity at the top of the cage being 120 W/m2. Animals were free to move within the cage with access to food and water ad libidum. Baseline electroretinography (ERG) readings and optical coherence tomography (OCT) images were collected prior to the light damage. After light exposure, animals were returned to normal light-dark cycle conditions (12 hours light, 174 lux and 12 hours darkness, <62 lux) for 24 hours, 1 week or 2 weeks (and for one group 3 months).

Tonabersat treatment of light damaged rats—LD rats were randomly assigned to low, middle or high dose of tonabersat (n=7 per group). The mixture of tonabersat in peanut butter was prepared fresh for each experiment. Three oral tonabersat doses were tested (n=7 per group) with follow up to 2 weeks post-injury, and the highest dose group then split with three animals taken for histological analysis, and four animals followed to 3 months post-injury. There was a ten-animal vehicle only control group. The group of rats that were kept for 3 months were maintained in separate cages for drug or vehicle-treated animals, but were kept under the same light conditions. Tonabersat was fed to animals in peanut butter at 0.26 mg/kg (on average 0.08 mg delivered, estimated circulating concentration 10 μM), 0.8 mg/kg (average of 0.24 mg delivered; 30 μM circulating) or 2.4 mg/kg (on average 0.72 mg delivered; 90 μM circulating) following consideration of previous unsuccessful human trial dosing levels. Silberstein S D. Tonabersat, a novel gap-junction modulator for the prevention of migraine. Cephalalgia. 2009; 29 Suppl 2:28-35; Dahlof C G, Hauge A W, Olesen J. Efficacy and safety of tonabersat, a gap-junction modulator, in the acute treatment of migraine: a double-blind, parallel-group, randomized study. Cephalalgia. 2009; 29 Suppl 2:7-16; Goadsby P J, Ferrari M D, Csanyi A, Olesen J, Mills J G, Tonabersat TONSG. Randomized, double-blind, placebo-controlled, proof-of-concept study of the cortical spreading depression inhibiting agent tonabersat in migraine prophylaxis. Cephalalgia. 2009; 29:742-50. Animals were fed immediately before the light exposure period; those which did not consume the drug were excluded from the study (total experimental, n=29 drug and vehicle treated animals). The treatment groups were known to the investigator at the time of treatment, but groups were then randomized prior to statistical comparison which meant that analysis was conducted without the knowledge of which were treated or control animals. In summary, adult rats were dark adapted overnight and the ERG data collected. Animals were fed the vehicle or tonabersat before intense light exposure. Two weeks later animals were again assessed with ERG and OCT and tissue collection proceeded immediately after, except for four rats in the high dose group that were under experimentation for 3 more months.

Hyperglycemic Rat Diabetic Retinopathy Model—A spontaneously hyperglycemic strain of SD rats, which developed clinical signs of diabetic retinopathy within 4 weeks of birth were identified and isolated within the Vernon Jansen Animal Research Unit, Faculty of Medical and Health Sciences at the University of Auckland. The identification of these rats presenting with hyperglycemia and microaneurysms provided an opportunity to treat a complex, chronic disease model and assess the treatment effect based upon objective, measurable endpoints, despite lacking precise information on the etiology of the disease. Inbreeding was carried out over three generations and eyes were screened for abnormalities between 4-8 weeks of age. Additional information about these rats is presented as supplementary information. Glucose levels were tested in non-fasting rats (Lee J J, Yi H Y, Yang J W, Shin J S, Kwon J H, Kim C W. Characterization of streptozotocin-induced diabetic rats and pharmacodynamics of insulin formulations. Biosci Biotechnol Biochem. 2003; 67:2396-401) using a Freestyle Optium Glucometer (Abbott Laboratories Ltd., UK) and Freestyle Optium glucose strips.

Ten rats per group were the selected (10 normal SD and 10 hyperglycemic) grown to 5 weeks of age and assessed using OCT and ERG. The hyperglycemic rat group was then split into two subgroups of five, with one subgroup fed tonabersat once daily for 14 days from weeks 5 to 7 at a low dose level of 0.28 mg/kg in 0.5 g peanut butter, with the other subgroup fed 0.5 g peanut butter only. All animals were assessed again using ERG and OCT at 8 weeks of age before euthanizing animals and removing the eyes for immunohistochemical analysis. Data groups were randomized prior to the statistical comparison. In summary, normal SD and hyperglycemic rats were analyzed with OCT and ERG at 5 weeks of age; the hyperglycemic rats were then split into two groups and fed the vehicle or tonabersat for 14 days during weeks 6 and 7 of age. At week 8, animals were again assessed with ERG and OCT and tissues collected for immunohistochemical analysis. Four tonabersat-treated rats were, however, left until 3 months of age before final ERG and OCT and tissue collection.

Evans Blue Dye Assessment of Vessel Leak—To investigate whether micro- and macro-aneurysms in the hyperglycemic rat retinas (observed using OCT) reflect sites of vessel leak, rats were perfused at the age of 3 months with Evans Blue dye as previously described. Cai S, Yang Q, Hou M, Han Q, Zhang H, Wang J, et al. Alpha-Melanocyte-Stimulating Hormone Protects Early Diabetic Retina from Blood-Retinal Barrier Breakdown and Vascular Leakage via MC4R. Cell Physiol Biochem. 2018; 45:505-22. Briefly, Evans Blue dye (30 mg/ml; Sigma-Aldrich, USA) was dissolved in normal saline and filtered. The dye was delivered as an injection into the rat-tail vein of normal SD and hyperglycemic rats at 45 mg/kg and allowed to circulate for 2 hours. Eyes were enucleated while rats were under deep anesthesia and an intracardial injection of 3M KCl was then administered rapidly to euthanize the animals. The posterior segment cups were fixed whole in 4% paraformaldehyde for 30 min and the retinas removed and laid flat. Evans Blue was stimulated at 559 nm wavelength and visualized by its red fluorescence emission using Olympus FluoView FV1000 (Olympus Corporation, Tokyo, Japan).

Electroretinogram Recording—The procedure was performed as described previously. Vessey K A, Wilkinson-Berka J L, Fletcher E L. Characterization of retinal function and glial cell response in a mouse model of oxygen-induced retinopathy. J Comp Neurol. 2011; 519:506-27. Essentially, SD rats were dark-adapted overnight for 12-14 hours before the ERG recording. For the dry AMD, the ERG baseline was recorded for all groups before light damage and at time points after the light damage insult (24 hours, 1 week, 2 weeks and 3 months post intense light). For the DR model, ERGs were recorded at 5 weeks of age to compare normal SD and hyperglycemic rat retinal function. Tonabersat treated and vehicle control hyperglycemic rats were assessed again at 8 weeks of age. After dark adaptation, rats were anaesthetized by an intraperitoneal injection of a combination of ketamine (75 mg/kg, Parnell Technologies, New Zealand) and domitor (0.5 mg/kg, Pfizer, New Zealand). A dim red light generated by a light-emitting diode (λmax=650 nm) was used during manipulations of dark-adapted animals. The corneas were maintained hydrated with 1% carboxymethylcellulose sodium (Celluvisc, Allergan, USA) during ERG recording. Right and left eye ERGs were recorded using gold ring electrodes (Roland Consult Stasche & Finger GmbH, Germany). A U-shaped active electrode was kept in contact with the center of the cornea. A V-shaped inactive electrode was hooked around the front teeth and in contact with the wet tongue. Normal body temperature was maintained to avoid temperature-driven ERG amplitude fluctuation by placing animals on a 37° C. heated pad. Full-field ERG responses were elicited by a twin-flash (0.8 ms second stimulus interval) generated from a photographic flash unit (Nikon SB900 flash, Japan), via a Ganzfeld sphere. An integrating sphere approximately 650 mm in diameter and painted white internally was used to reflect the flash light onto the entire retina. The flash intensity range was from −2.9 to 2.1 log cd·s/m2 and was attenuated using neutral density filters (Kodak Wratten, Eastman Kodak, USA), to obtain light intensities of −3.9, −2.9, −1.9, 0.1, 1.1, 1.6, 1.8 and 2.1 log cd·s/m2. The flash intensity was calibrated using an IL1700 research radiometer (UV Process Supply Inc., USA). This study utilized a twinflash paradigm for the isolation of rod and cone pathways. Paired flashes of identical luminous energy were triggered from the flash unit. The rod and cone mixed responses were recorded after the initial flash, and the response from the second flash was recorded and represents the function from the cones only. The rod PIII response was derived through digital subtraction of the cone response from the initial mixed response. The PIII component of the ERG is a direct reflection of rod photocurrent and the slope of the a-wave is more appropriately interpreted by taking into account the information about the photocurrent of rods after fitting their response to a computational model. For that, ERG data at the highest light level is fitted with a model of rod response assuming an initially linear rise of response amplitude with intensity, followed by saturation to reveal the PII (the bipolar cell component) and PIII (the photoreceptor component). Through this rod PII and PIII isolation, we can confirm that a-wave and b-wave ERG data correspond to alterations in both cone and rod pathways. The oscillatory potentials (OPs) are another way of investigating inner retinal function. OPs were isolated by subtracting the raw bwave from the rod PII. Weymouth A E, Vingrys A J. Rodent electroretinography: methods for extraction and interpretation of rod and cone responses. Prog Retin Eye Res. 2008; 27:1-44. The summed amplitude of OPs 2, 3, and 4 were analyzed. Recordings were performed in a Faraday cage to reduce electrical noise. The results of ERG signals were amplified 1,000 times by a Dual Bio Amp (AD Instruments, Australia) and waveforms were recorded using Scope software (AD Instruments, New Zealand) and analyzed using published algorithms of the amplitudes of a-wave and b-wave of each eye. Guo C X, Mat Nor M N, Danesh-Meyer H V, Vessey K A, Fletcher E L, O'Carroll S J, et al., supra; Vessey K A, Wilkinson-Berka J L, Fletcher E L, supra. To achieve 80% power, and with an alpha value of 5%, we have determined ERG studies need a sample size of 5.

Optical Coherence Tomography—Spectral domain optical coherence tomography (SD-OCT; Micron IV; Phoenix Research Laboratories, USA) was employed to obtain information on in vivo retinal layer morphology. OCTs were executed immediately after ERG recordings had been taken, with the animals under anesthesia and with pupils dilated using 1% Tropicamide (Bausch & Lomb New Zealand Ltd., New Zealand). Guo C X, Mat Nor M N, Danesh-Meyer H V, Vessey K A, Fletcher E L, O'Carroll S J, et al., supra. Rats were placed on a 37° C. heating pad to maintain body temperature and to prevent the development of cold cataracts. Eyes were covered with Poly Gel (containing 3 mg/g Carbomer; Alcon Laboratories Pty Ltd, Australia) and the retina was imaged by contacting the OCT lens to the gel. StreamPix 6 software, version 7.2.4.2 (Phoenix Research Laboratories, USA) was used for image acquisition. The SD-OCT horizontal line B-scan had 2 μm axial resolution, and consisted of 1024 pixels per A-scan. Ten B-scans acquired 2 mm from the optic nerve in the dorsal retina were taken and averaged. Images were analyzed using InSight software, version 1.1.5207 (Phoenix Research Laboratories, USA). Choroidal layer thickness was measured from the hyper-reflective Bruch's membrane to the choroidal-scleral interface. Outer nuclear layer (ONL) thickness was measured from the outer limiting membrane (OLM) to the outer plexiform layer (OPL) interface.

Tissue Collection and Processing—At the end of the final OCT recording rats was deeply anesthetized using a combination of ketamine (75 mg/kg, Parnell Technologies, New Zealand) and domitor (0.5 mg/kg, Pfizer, New Zealand). Animals were perfused transcardially with saline for 2-3 min followed by 30 min perfusion with 4% paraformaldehyde in a 0.1 M phosphate buffer, pH 7.4 (PB). Eyes were dissected from the orbit and the eyecups further immersion fixed in 4% paraformaldehyde, 30 min before washing in PB. Tissues were then cryo-protected by taking them though 10% and 20% sucrose/PB solutions at room temperature for 30 min each, before soaking in 30% sucrose/PB at 4° C. overnight. The tissue was then embedded in optimum cutting temperature compound (Sakura Finetek, Torrance, USA) for cryosectioning (16 μm section thickness) in the vertical plane using a Leica CM3050 S cryostat (Leica, Germany). Sections were collected on Superfrost Plus slides (Labserv, New Zealand) for immunohistochemical labelling. For the DR animals we collected spleen, pancreas, liver, heart and kidneys from randomly selected vehicle-injected animals (see supplementary information).

Immunohistochemical Labelling of Tissue Sections—Frozen tissue sections were air-dried at room temperature for 10-15 min and washed with 0.1 M PB. Sections were encircled with a PAP pen (Invitrogen, New Zealand) to form incubation wells and blocked with 6% normal goat serum or donkey serum (Invitrogen, USA), 1% bovine serum albumin (BSA) and 0.5% Triton X-100 in 0.1 M PB for 1 hour at room temperature. Primary antibodies included rabbit anti-Connexin43 (1:1000, Cat C6219, Sigma Aldrich, USA), mouse anti-Iba-1 expressed specifically by microglial cells (ionized calcium-binding adapter molecule 1, 1:250, Cat Ab5076, Abcam, USA), and mouse anti-GFAP for astocytes and activated Müller cells (1:1000, Cat C9205, Sigma-Aldrich, USA) antibodies. Sections were incubated with primary antibody overnight at room temperature and then washed four times for 15 min each in 0.1 M PB. The secondary antibody, goat anti-rabbit or goat anti-mouse conjugated with Alexa™ 488 or Alexa™ 594 (Invitrogen, Australia), was diluted at 1:500 and applied for 2-3 hours in the dark at room temperature. The slides were then washed thoroughly with 0.1 M PB and cell nuclei stained with DAPI (1:1000; Sigma-Aldrich, USA) before cover slipping with anti-fading medium (Citifluor Ltd, UK). Coverslips were sealed with nail polish. Sections were imaged using an Olympus FluoView FV1000 confocal laser scanning microscope fitted with 405, 473 and 559 nm wavelength excitation lasers (Olympus Corporation, Japan).

Statistical analysis—Graphing and statistical analyses were performed using GraphPad Prism 5 (GraphPad Software, USA). All data is presented as the mean±the standard error of the mean (SEM). Functional and morphological data were compared using analysis-of-variance (ANOVA) with an alpha value of 0.05. A two-way ANOVA followed by a Bonferroni post-test was used in the ERG response analysis to compare the effect of stimulus intensity. A one-way ANOVA followed by Tukey's test was used in the ERG response at the intensity of 2.1 log cd·s/m2 in control and light-damage animals and also in the OCT data analysis. Statistical analysis of rod PII and PIII was performed using an unpaired t-test with a Welch's correction, assuming the distribution of means across samples was normal.

### Example 2

### Treatment with Hemichannel Blockers Preserved Choroidal Thickness, Retinal Thickness and Rescued Retinal Function in Animal Bright-Light Damage Model of Retinal Degradation

Mixed a-wave ERG data, plotted for the range of light intensities tested, results in a negative deflection, increasing with flash intensity from lower to mid-range levels. The mixed b-wave ERG response is a positive deflection and is consistent for most flash intensities.

Following 24 hours of light exposure, ERG responses of the albino rats were significantly attenuated with a maximum a wave amplitude of −100 V. ERG data are shown in FIG. 1 for the vehicle-fed group of animals at 2 weeks post-injury and for each of the three treatment doses at 24 hours, 1 week and 2 weeks post-light damage.

At 24 hours post-light exposure, there were no differences between the vehicle control group and any of the three tonabersat dose groups (FIG. 1).

However, significant improvements in mixed a-wave amplitude in both 0.26 mg/kg and 0.8 mg/kg treated animals compared to the light damaged control group were seen at 1-week post treatment (p<0.01; FIGS. 1B-C) and at a wider range of intensities: 0.1-2.1 log cd·s/m2, in the 2.4 mg/kg treatment group (p<0.001; FIG. 1D).

By 2 weeks post-treatment all three doses of the oral tonabersat hemichannel blocker gave a significant recovery in mixed a-wave amplitude (p<0.001, FIGS. 1B-D) at intensities 0.1-2.1 log cd·s/m2. These animals had an almost 500 V improvement in ERG a-wave at the highest intensity employed over the vehicle treated light-injured group (p<0.001; FIGS. 1B-D), with treatment causing recovery of the ERG function only slightly less than the SD rat average of about −600 V. In other words, all three doses of the hemichannel blocker restored ERG function.

There was significant improvement in mixed b-wave function for 0.26 and 0.8 mg/kg tonabersat-treated groups 24 hours after treatment (FIGS. 1E-F), not seen in the high dose treatment group (FIG. 1G). However, an overt improvement in inner retinal function was seen as increased mixed b-wave amplitude throughout all stimulus intensities by 1 and 2 weeks post-treatment for all three doses of tonabersat (FIGS. 1E-G). The highest dose of tonabersat, 2.4 mg/kg, showed the highest improvement (on average 1200 V) which is in the normal range for undamaged SD rats in the absolute amplitude of the b-wave [36]. For 0.26 and 0.8 mg/kg there was an improvement (on average 1000 V) in the absolute amplitude of the b-wave. Nonetheless, at the end of the 2-week recovery period all tonabersat-treated animal mixed a-wave and mixed bwave functions were within the range of variance for normal, undamaged albino rats. Heiduschka P, Schraermeyer U., Comparison of visual function in pigmented and albino rat by electroretinography and visual evoked potentials. Graefes Arch Clin Exp Ophthalmol. 2008; 246:1559-73. In other words, all three doses of the hemichannel blocker rescued inner retinal function.

A cohort of 4 animals treated with the highest dose of oral tonabersat (2.4 mg/kg) was maintained for 3 months under normal breeding and food access conditions. The benefit of the orally administered tonabersat treatment was maintained long term. The ERG a-wave and b-wave wavelength amplitudes in the original 7 treated animals assessed at 2 weeks following oral tonabersat (2.4 mg/kg) were only slightly lower compared to the cohort of 4 treated animals taken through and assessed 3 months after orally administered tonabersat. In the a-wave there was an improvement in photoreceptor function exceeding 400 V and in the b-wave the amplitude exceeded 800 V compared with vehicle treated controls (FIG. 2A-B). In the PIII rod and PII rod response, there was no change 3 months after oral tonabersat treatment compared to before light damage, suggesting that tonabersat treatment had completely preserved photoreceptor function. In contrast, changes in rod PIII and rod PII amplitudes were significantly reduced in the vehicle only-treated light-damaged group at 3 months (p<0.001; FIGS. 2C-D). In other words, all three doses of the hemichannel blocker rescued photoreceptor function, and the PIII rod and PII rod responses.

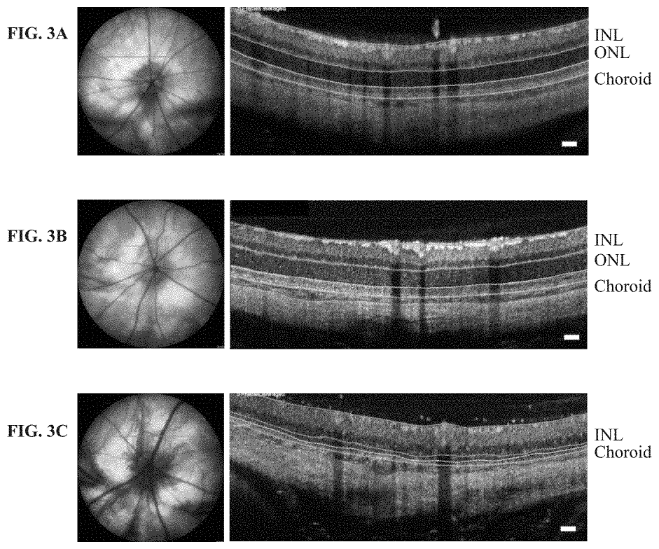

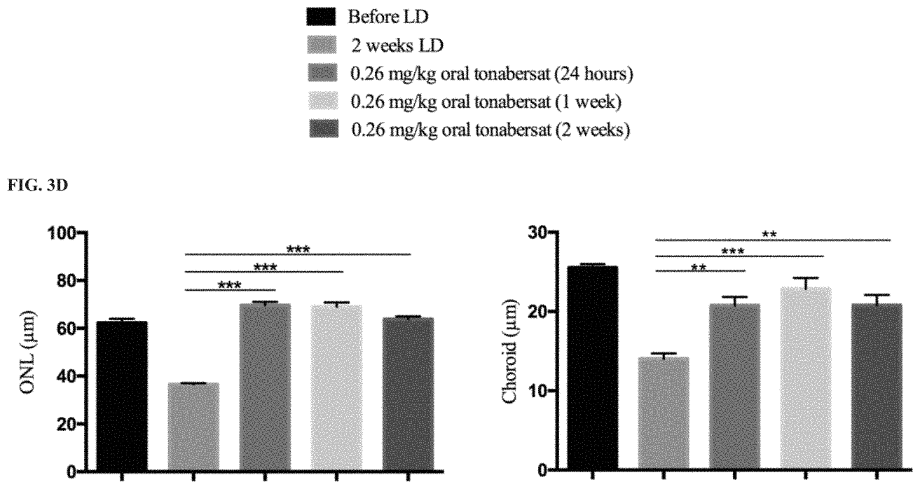

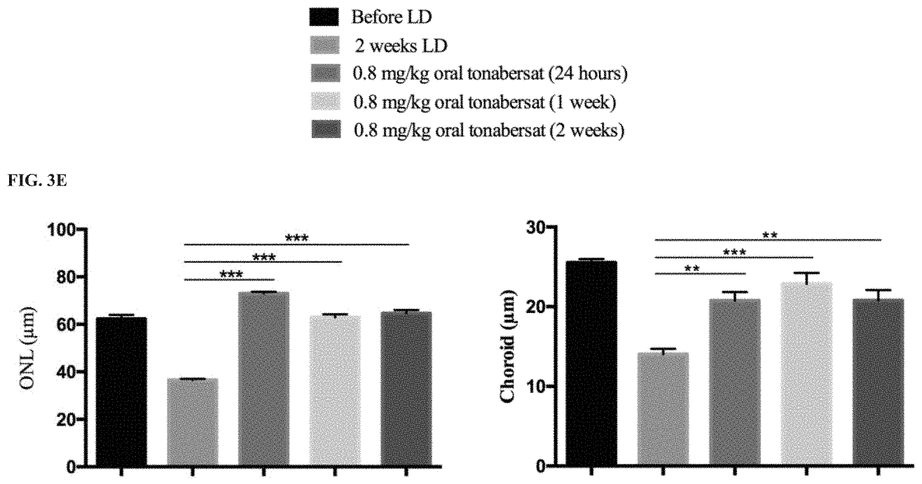

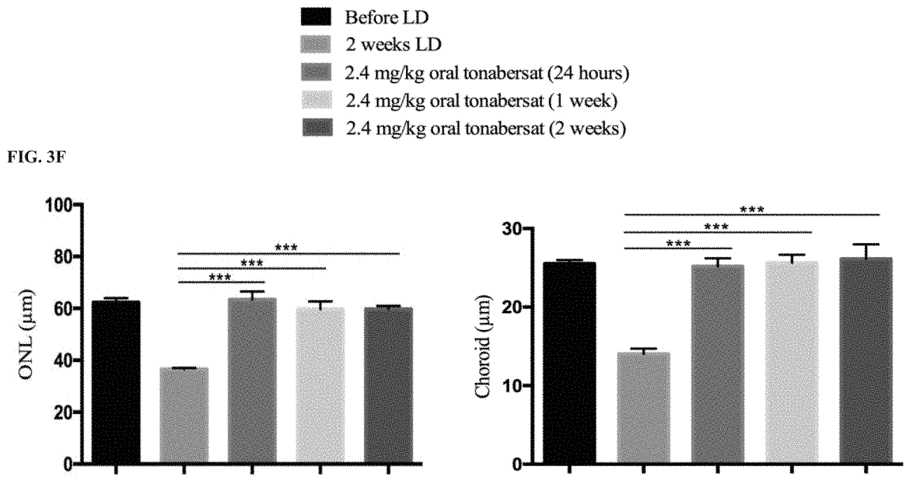

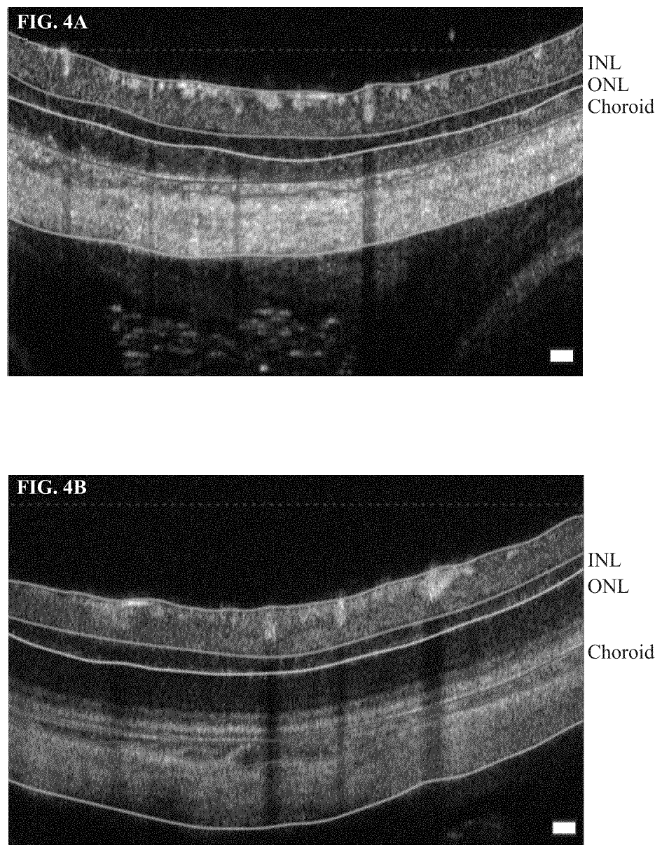

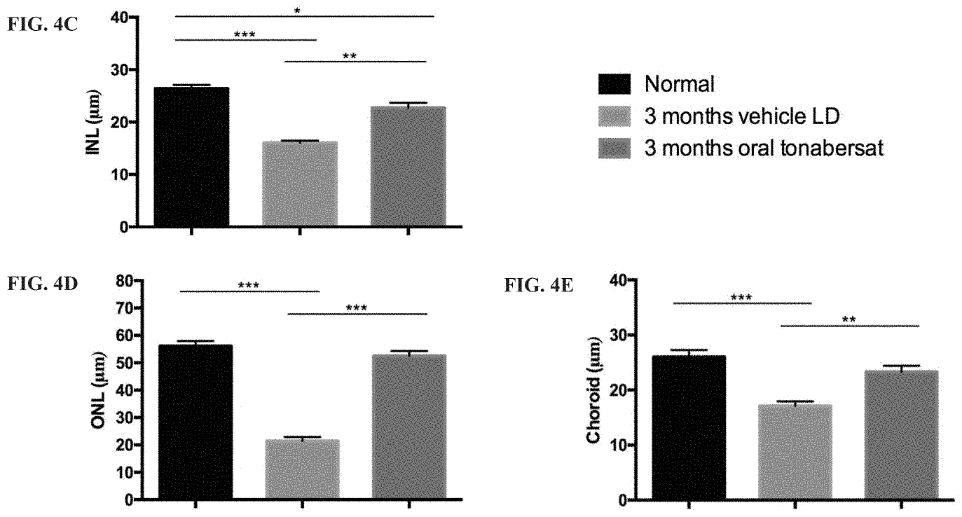

Analysis of retinal layers and choroidal thickness was conducted using OCT scans at 24 hours, 1 week and 2 weeks post-injury. FIG. 3 shows the typical appearance of the fundus and OCT scan for a normal adult Sprague Dawley rat, vehicle-treated light-damaged rat, and 2.4 mg/kg tonabersat-treated animal 2 weeks after light damage (FIG. 3A-C). There was significant thinning of both the retina and choroid in vehicle treated animals evident at the 2-week time point compared to the same eyes prior to light damage (p<0.001; FIG. 3A-B). The loss of retinal thickness was primarily owing to thinning of the ONL. However, all three doses of oral tonabersat significantly preserved both retinal and choroidal thickness, with no thinning detected at any of the post-treatment time points (24 hours, 1 week and 2 weeks) investigated (FIG. 3 D-F). OCT analysis 3 months post treatment following 2.4 mg/kg orally administered tonabersat showed both the retinal and choroidal thicknesses were significantly preserved compared to the vehicle treatment group (p<0.001; FIGS. 4A-B). Retinal thinning was evident in both the inner nuclear layer (INL; FIG. 4C) and ONL (FIG. 4D) layers in vehicle treated animals but there was no difference in ONL thickness in oral tonabersat-treated animals compared to the same animals prior to the light damage procedure. There was a slightly reduced thickness of the INL in the 3 months oral tonabersat treated group compared to the same animals imaged prior to the light damage procedure (p<0.05). The vehicle treated rats showed significant INL thinning compared to the same retinas prior to the light damage procedure (p<0.001). There was no difference in choroidal thickness between oral tonabersat treated rats at 3 months and the same rats assessed prior to the light damage procedure (FIG. 4E). In contrast, vehicle treated light damaged rats assessed at 3 months post-injury had significant thinning of the choroid (p<0.001). In other words, all three doses of the hemichannel blocker rescued both retinal structure and choroidal structure.

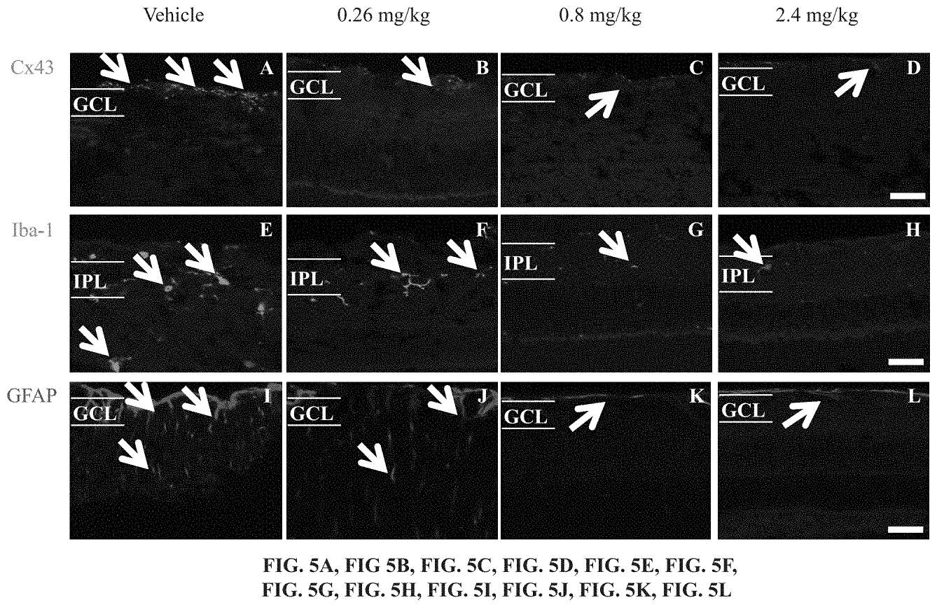

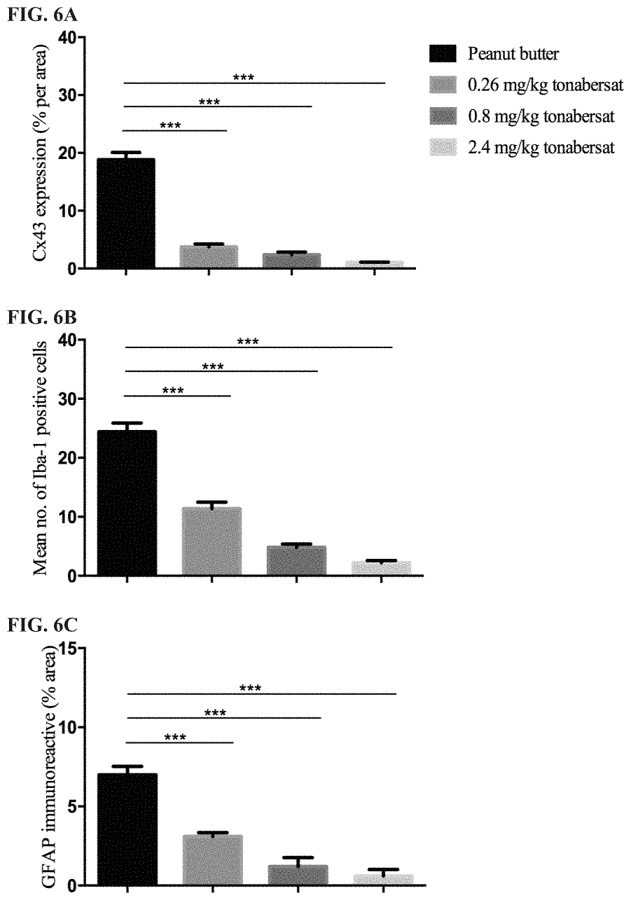

Following final ERG and OCT assessments, eyes were removed and the posterior segment of the eyes containing the retina and the attached RPE-choroid-sclera assessed using immunohistochemical labelling of GFAP to investigate the extent of gliosis (astrocytosis), Iba-1 to determine the microglia immunoreactivity pre- and post-treatment, and Connexin43. Compared to vehicle treated rats (FIG. 5A), tonabersat treated rats showed less Connexin43 immunoreactivity in the retina at all three tonabersat doses used (FIGS. 5B-D). Iba-1 immunolabelled cells were less active compared to vehicle treated (FIG. 5E) in the inner plexiform layer (IPL) of the retina in the drug treated groups (FIG. 5F-H). A slightly higher level of Iba-1 reactivity was seen in the 0.26 mg/kg treated rats. GFAP immunoreactivity did not increase in the retina of 0.8 mg/kg tonabersat (FIG. 5K) and 2.4 mg/kg tonabersat (FIG. 5L) compared to vehicle (FIG. 51) treated rats. In the 0.26 mg/kg dosed animals, there was a slight increase in GFAP labelling (FIG. 5J) which was significantly less than that seen in vehicle treated rats. Image quantification showed significantly less GFAP, Connexin43 and Iba-1 levels in all tonabersat treated groups compared with the vehicle controls (p<0.001) (FIGS. 6A-C), with a tendency to dose response (higher doses more effective at maintaining normal levels of these retinal inflammation markers).

### Example 3

### Treatment with Hemichannel Blockers Rescued Retinal Function in Hyperglycemic Animals with Diabetic Retinopathy

The average body weight of control SD rats was 185±1.1 g at 4 weeks of age, 198.2±0.8 g at 6 weeks and 217.5±1.3 g at 8 weeks. The hyperglycemic rat had a lower body weight with 172.5±2.5 g at 4 weeks of age, 179.6±2.1 g at 6 weeks, and 183.1±1.8 g at 8 weeks. The difference between all three age groups compared to age matched normal SD rats was statistically significant (t-test, p<0.001). Blood glucose readings in normal SD rats ranged from 4.9-7.4 mmol/L (average 6.07 mmol/L with no significant difference between age groups); in the hyperglycemic rats glucose levels were 14.0-21.0 mmol/L with the average being 16.85±0.63 mmol/L at 4 weeks, 15.43±0.79 mmol/L at 6 weeks and 16.54±0.65 mmol/L at 8 weeks, the level of hyperglycemia remaining consistent from a young age. The difference between hyperglycemic rats all three age groups and normal SD rats was statistically significant (t-test, p<0.001).

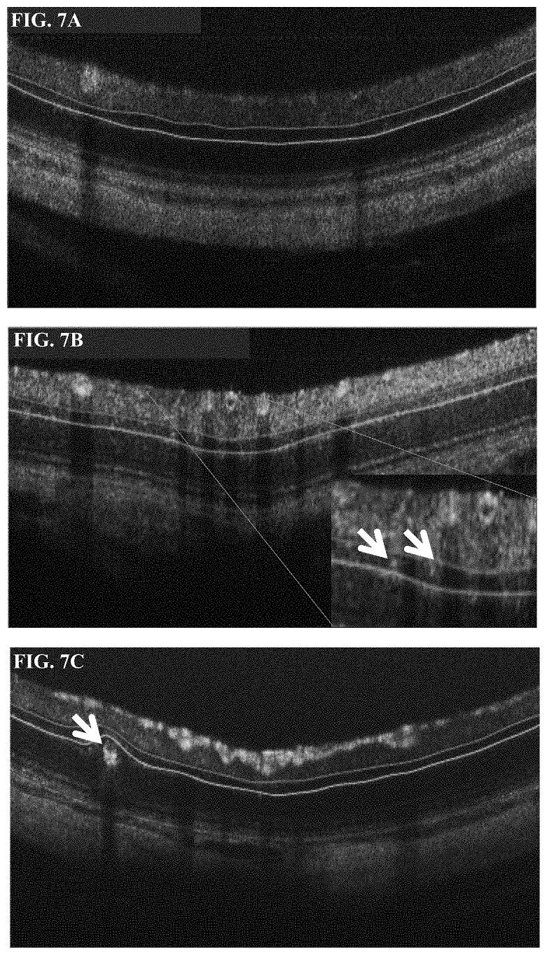

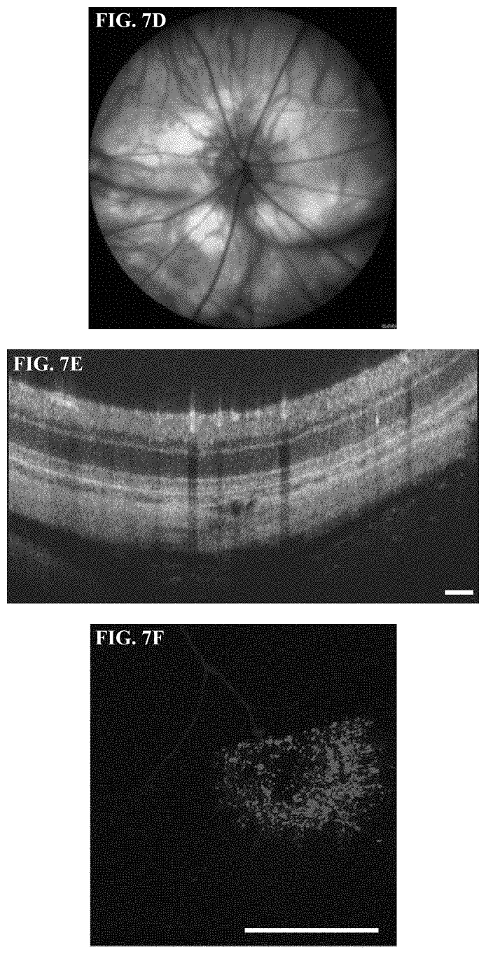

Slit lamp examination of the anterior segment (cornea, lens) of hyperglycemic rats at 4 weeks of age did not reveal any obvious macroscopic differences compared to SD rats from which this strain was derived. The cornea, lens and iris appeared to be identical to normal SD rats, and there were no signs of diabetic cataract or neovascularization. However, OCT of the hyperglycemic rats revealed an average of 5-8 hyperreflective spots per eye (based upon 7 evenly spaced OCT scans across the retina and therefore an underestimate for the whole eye), but no more than 1-2 in normal SD rats. The hyperreflective spots appeared to be microaneurysms (20-30 μm diameter) and macroaneurysms (140-160 μm) (FIGS. 7B-C) and they were located specifically in the INL and ONL. There were no significant changes in retinal or choroidal thickness, although the choroid appeared to be slightly reduced in thickness in animals with aneurysms. Evans blue dye perfusion confirmed blood vessel leakage at sites of the aneurysms mapped using OCT (FIG. 7F).

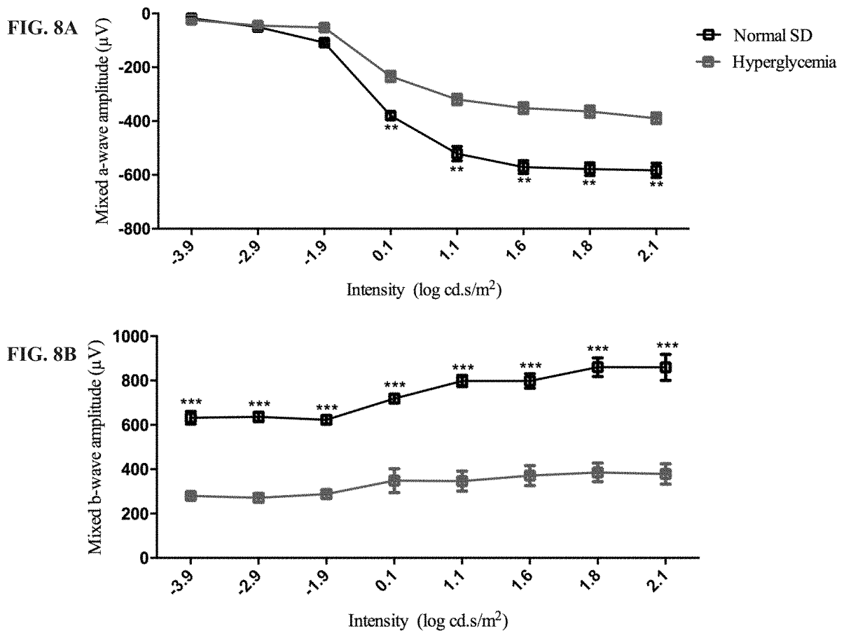

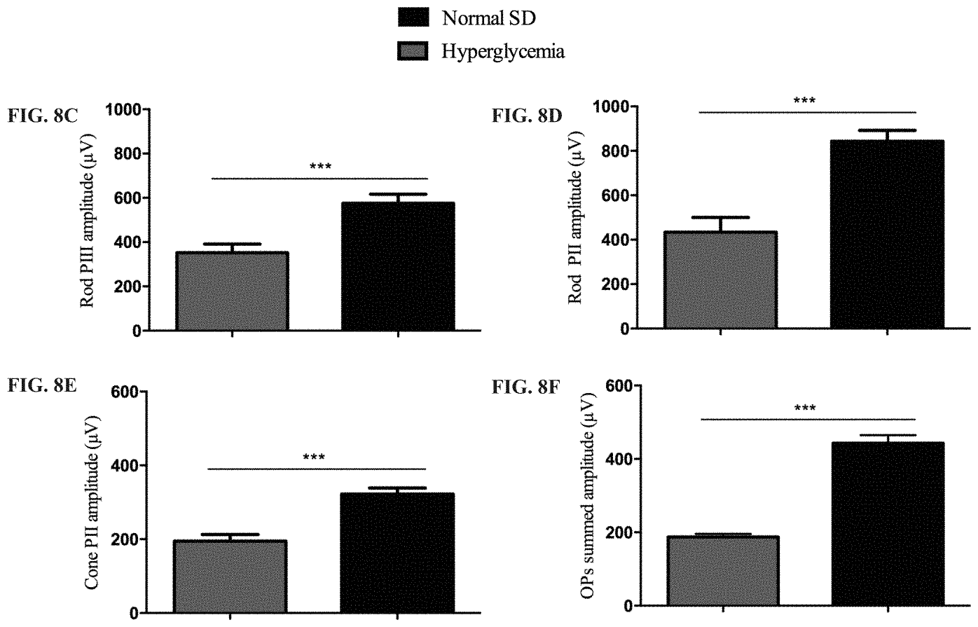

To determine whether the aneurysms affected retinal function, ERG analysis was carried out at 5 weeks of age to compare hyperglycemic rats retinal function with that of normal SD rats. Representative ERG waveforms are shown in FIG. 8. The average mixed a-wave amplitude was significantly reduced in hyperglycemic rats compared to normal SD rats for intensities 0.1-2.1 log cd·s/m2 (p<0.01), with SD rats measuring −630 V at the highest intensity, compared to −370 V in the hyperglycemic diabetic rat retina. Mixed ERG b-wave amplitude was also significantly reduced in hyperglycemic rats for intensities −3.9-2.1 log cd·s/m2 (p<0.001), with normal SD rats showing a maximum intensity of 800 V but only 400 V in diabetic animals. There was no difference between a-wave and b-wave implicit times. Further analysis revealed amplitudes were significantly reduced in the hyperglycemic rats for rod PIII (p<0.001), PII (p<0.001), cone PII (p<0.001) responses and for Oscillatory Potentials (OPs) summed amplitude (p<0.001) (FIG. 8). There were no significant changes in rod PIII sensitivity or rod PII, cone PII and OPs summed implicit times.

For the tonabersat DR treatment arm, ten hyperglycemic rats grown to week 5 were split into two equal groups with one group fed tonabersat once daily for 14 days from weeks 5 to 7 at a low dose level of 0.28 mg/kg. At week 8 there was no difference in body weight between treated and untreated hyperglycemic rats, and no significant difference in retinal layer thickness (INL or ONL) or the thickness of the choroid. However, there were differences in the number and size of microaneurysms after treatment (FIG. 9A-B). Retinal function had significantly recovered in drug-treated hyperglycemic rats compared to vehicle-injected hyperglycemic rats (FIG. 9C). In treated animals mixed ERG a-wave was significantly higher at intensities 0.1-2.1 cd·s/m2 (p<0.001), maximum intensity −630 V compared to −370 V in untreated animals at 8 weeks, and closely matching undamaged control SD rat levels (FIG. 8A). Similarly, mixed b-wave signals had significantly recovered in the tonabersat treated animals for all intensities (p<0.001), with maximum intensity of 700 V compared to only 400 V at this time point in the untreated controls (FIG. 9D), and again had recovered to near normal SD rat mixed b-wave values (Cf. FIG. 9B). Implicit times were not different. Further analysis revealed that treated hyperglycemic rats treated with a hemichannel blocker compound had significantly recovered rod PIII (p<0.001), PII (p<0.001), cone PII (p<0.01) and summed OPs (p<0.01) amplitudes (FIG. 9E-H). There was no difference in implicit times between pre- and post-treatments. In other words, the hemichannel blocker recovered and rescued retinal function and structure.

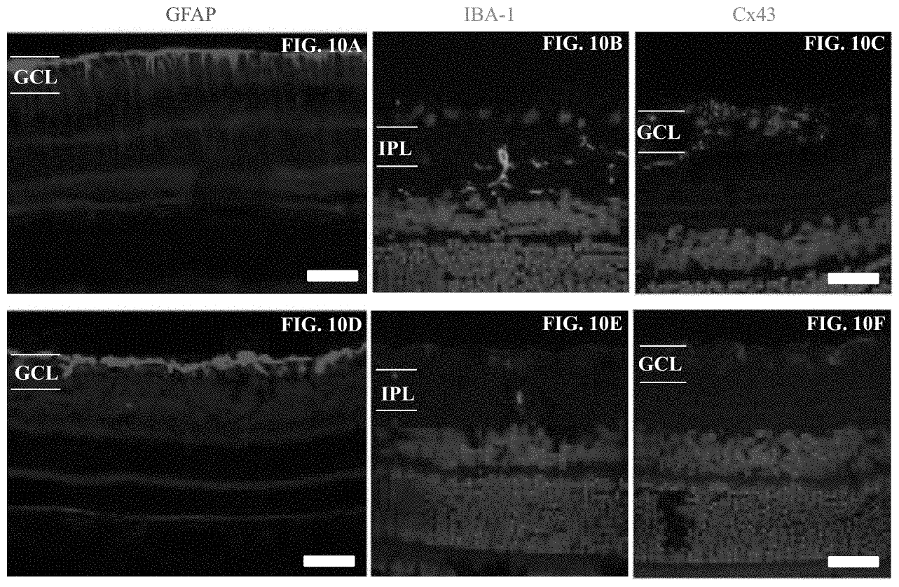

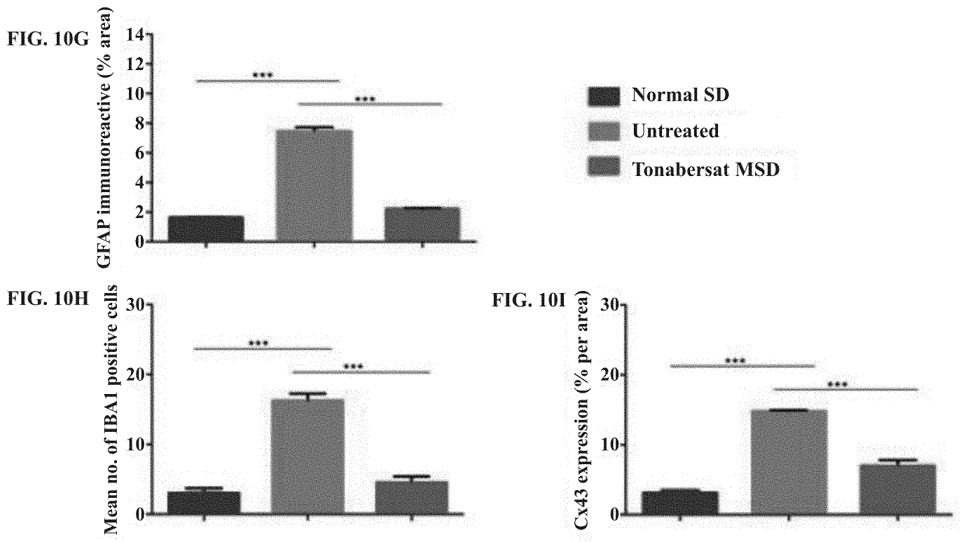

To determine whether the differences seen with OCT and ERG in the hyperglycemic rats correlated with retinal inflammation, eyes were removed for immunohistochemical examination at 8 weeks. GFAP labelling was intense in the retinal ganglion cell (RGC) layer and gliosis was visible in areas around microaneurysms in the hyperglycemic rat retinas, and extended from the nerve fiber layer (NFL) to the ONL indicating Müller cell activation (FIG. 10A). There was abnormally high Iba-1 labelling in the hyperglycemic retinas (FIG. 10B) indicating activated microglia in the inner retinal layers where cells with enlarged soma and numerous elongated branches were present, and Connexin43 labelling was abnormally high in the GCL of hyperglycemic rats (FIG. 10C). Hyperglycemic rats that had been dosed with tonabersat daily for 14 days had reduced inflammation as evident by reduced labeling for all three markers (FIG. 10E-F). Quantification of the results are shown in FIG. 10G, with all three markers, GFAP, Connexin43 and Iba-1 significantly higher in untreated hyperglycemic rats compared to undamaged control retinas (p<0.001), and all three treated groups recovering to normal at 8 weeks of age, and showing significantly less labeling than untreated rat retina levels (p<0.001).

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, and in embodiments or examples of the present invention, any of the terms “comprising”, “consisting essentially of”, and “consisting of” may be replaced with either of the other two terms in the specification. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A method for rescuing or restoring retinal function in a subject having a chronic retinal disorder, comprising administering an effective amount of a hemichannel blocker to said subject.

2. A method for rescuing or restoring retinal structure in a subject having a chronic retinal disorder, comprising administering an effective amount of a hemichannel blocker to said subject.

3. A method for rescuing or restoring choroidal function in a subject having a chronic retinal disorder, comprising administering an effective amount of a hemichannel blocker to said subject.

4. A method for rescuing restoring choroidal structure in a subject having a chronic retinal disorder, comprising administering an effective amount of a hemichannel blocker to said subject.

5. The method of any of claims 1-3 or 4, wherein the hemichannel blocker is a connexin 43 hemichannel blocker.

6. The method of any of claims 1-3 or 4, wherein the hemichannel blocker is a small molecule hemichannel blocker.

7. The method of any of claims 1-3 or 4, wherein the hemichannel blocker is N-[(3S,4S)-6-acetyl-3-hydroxy-2,2-dimethyl-3,4-dihydrochromen-4-yl]-3-chloro-4-fluorobenzamide (tonabersat).

8. The method of claim 6, wherein the small molecule hemichannel blocker is of Formula (I) or Formula (II): 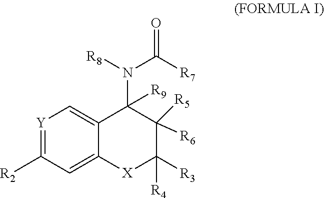
wherein Y is C—R₁;
R₁ is acetyl;
R₂ is hydrogen, C₃₋₈ cycloalkyl, C₁₋₆alkyl optionally interrupted by oxygen or substituted by hydroxy, C₁₋₆alkoxy or substituted aminocarbonyl, C₁₋₆alkylcarbonyl, C₁₋₆alkoxycarbonyl, C₁₋₆ alkylcarbonyloxy, C₁₋₆alkoxy, nitro, cyano, halo, trifluoromethyl, or CF₃S; or a group CF₃-A-, where A is —CF₂—,
—CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆alkoxycarbonylamino, C₁₋₆alkyl-thiocarbonyl, C₁₋₆alkoxy-thiocarbonyl, C₁₋₆alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆alkoxysulphinylamino or C₁₋₆alkoxysulphonylamino, or ethylenyl terminally substituted by C₁₋₆alkylcarbonyl, nitro or cyano, or —C(C₁₋₆alkyl)NOH or —C(C₁₋₆alkyl)NNH₂; or amino optionally substituted by one or two C₁₋₆alkyl or by C₂₋₇ alkanoyl; one of R₃ and R₄ is hydrogen or C₁₋₄alkyl and the other is C₁₋₄alkyl, CF₃ or CH₂X^{a} is fluoro, chloro, bromo, iodo, C₁₋₄alkoxy, hydroxy, C₁₋₄alkylcarbonyloxy, —S—C₁₋₄alkyl, nitro, amino optionally substituted by one or two C₁₋₄alkyl groups, cyano or C₁₋₄alkoxycarbonyl; or R₃ and R₄ together are C₂₋₅ polymethylene optionally substituted by C₁₋₄alkyl;
R₅ is C₁₋₆alkylcarbonyloxy, benzoyloxy, ONO₂, benzyloxy, phenyloxy or C₁₋₆alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄alkyl, cyano, azido, C₁₋₄alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C₁₋₆ alkyl amino, amino-C₁₋₆ alkyl, hydroxy-C₁₋₆ alkyl, halo-C₁₋₆ alkyl, C₁₋₆ acyloxy-C₁₋₆ alkyl, C₁₋₆alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆alkyl;
or Formula II 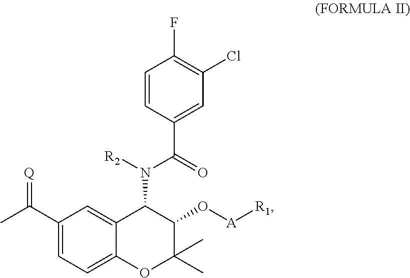
wherein
Q is O or an oxime of formula ═NHOR₄₃, wherein R₄₃ is (i) selected from H, C₁₋₄ fluoroalkyl or optionally substituted C₁₋₄ alkyl, or (ii) -A₃₀₀-R₃₀₀, wherein A₃₀₀ is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₃₀₀, R₃ and R₄ are selected independently from H, fluoro, C₁₋₄ alkyl, or C₁₋₄ fluoroalkyl, or R₃ and R₄ together with the atom to which they are attached form a cyclopropyl group, and R₃₀₀ is selected from groups [1], [2], [2A], [3], [4], [5] or [6];
R₂ is H,
A is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₁, R₃ and R₄ are selected independently from H, fluoro, C₁₋₄ alkyl, or C₁₋₄ fluoroalkyl, or R₃ and R₄ together with the atom to which they are attached form a cyclopropyl group,
R₁ is selected from groups [1], [2], [2A], [3], [4], [5] and [6] wherein the atom marked ** is directly connected to A: 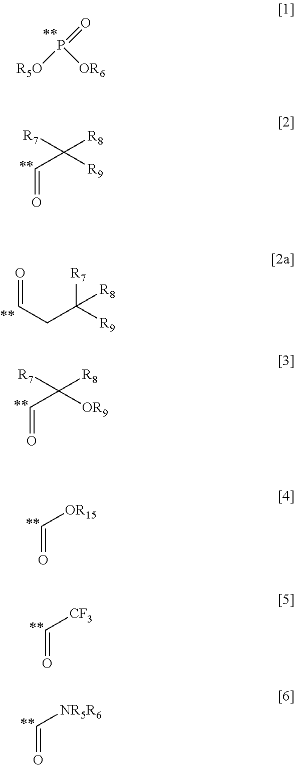
and R₆ are each independently selected from H, C₁₋₄ alkyl, C₁₋₄ fluoroalkyl, and benzyl;
R₇ is independently selected from H, C₁₋₄ alkyl, and C₁₋₄ fluoroalkyl;
R₈ is selected from:
(i) H, C₁₋₄ alkyl, or C₁₋₄ fluoroalkyl, or
(ii) the side chain of a natural or unnatural alpha-amino acid, or a peptide as described herein, or
(iii) biotin or chemically linked to biotin;
R₉ is selected from H, —N(R₁₁)(R₁₂), or —N⁺(R₁₁)(R₁₂)(R₁₃)X⁻, or —N(R₁₁)C(O)R₁₄ wherein R₁₁, R₁₂, and R₁₃ are independently selected from H, C₁₋₄ alkyl, or C₁₋₄ fluoroalkyl,
R₁₄ is H, C₁₋₄ alkyl, or C₁₋₄ fluoroalkyl,
R₁₅ is independently selected from C₁₋₄ alkyl and C₁₋₄ fluoroalkyl, and
X⁻ is a pharmaceutically acceptable anion.

9. The method of any of claims 1-3 or 4, wherein the hemichannel blocker is administered orally in amount ranging from about 10 to 200 mg per day.

10. The method of claim 7, wherein the hemichannel blocker is administered orally in an amount ranging from about 10 to about 200 mg per day.

11. The method of claim 7, wherein the hemichannel blocker is administered orally in an amount ranging from about 0.2 mg/kg to about 5 mg/kg.

12. The method of claim 7, wherein the circulating concentration of tonabersat in the subject ranges from about 10 micromolar to about 90 micromolar.

13. The method of claim 1, wherein said hemichannel blocker is administered by injection.

14. The method of claim 1, wherein said hemichannel blocker is administered orally.

15. The method of claims 1-3 or 4, wherein the hemichannel blocker is administered once per day.

16. The method of claims 1-3 or 4, wherein the hemichannel blocker is administered once per week.

17. The method of claim 7, wherein the subject is a human.

18. The method of claim 1, wherein the hemichannel blocker is not in a composition comprising a microparticle.

19. The method of claim 1, wherein the retinal function is selected from: mixed a-wave function, mixed b-wave function, and/or PII and PIII rod and cone function.

20. A method of improving retinal structural integrity in a subject having a chronic retinal disorder, comprising administering an effective amount of a hemichannel blocker to said subject.

21. The method of claim 20, wherein the retinal pigment epithelium is recovered.

22. The method of claim 20, wherein the retinal vascular endothelium is recovered.

23. The method of claim 20, wherein the normal retinal layer structure is recovered.

24. A method of reducing or eliminating micro- and/or macro-aneurysms in a subject having a chronic retinal disorder, comprising administering an effective amount of a hemichannel blocker to said subject.

25. A method of improving photoreceptor function in a subject having a chronic retinal disorder, comprising administering an effective amount of a hemichannel blocker to said subject.

26. A method of improving choroidal structural integrity in a subject having a chronic retinal disorder, comprising administering an effective amount of a hemichannel blocker to said subject.

27. The method of claim 26, wherein the choroidal thickness is recovered.

28. The method of claim 26, wherein the choroidal vascular bed is recovered.

29. A method of improving the choroidal vascular blood flow to the outer retina in a subject having a chronic retinal disorder, comprising administering an effective amount of a hemichannel blocker to said subject.

30. A method of improving choroidal blood flow in a subject having a chronic retinal disorder, comprising administering an effective amount of a hemichannel blocker to said subject.

31. A method for increasing survival of retinal function in a subject in need thereof, comprising administering to said subject a survival-promoting amount of N-[(3S,4S)-6-acetyl-3-hydroxy-2,2-dimethyl-3,4-dihydrochromen-4-yl]-3-chloro-4-fluorobenzamide (Xiflam).

32. The method of claim 31, wherein the survival-promoting amount is 10 to 200 mg per day.

33. The method of claim 31, wherein the survival-promoting amount is 20 to 100 mg per day.

34. The method of claim 31, wherein the increasing survival treats a chronic retinal disorder.

35. The method of claim 34, wherein the chronic retinal disorder is diabetic retinopathy.

36. The method of claim 34, wherein the chronic retinal disorder is diabetic macular edema.

37. The method of claim 34, wherein the chronic retinal disorder is selected from the group consisting of wet age-related macular degeneration, dry age-related macular degeneration, geographic atrophy and hypertensive retinopathy.

38. The method of claim 34, wherein the chronic retinal disorder is caused by retinal degeneration, edema, diabetes, ischemic retinal degeneration, retinal vascular occlusion, and central retinal vein occlusion.

39. The method of claim 31, wherein mixed a-wave function and/or improved mixed b-wave function are improved.

40. The method of claim 31, wherein PII and PIII rod and cone function are improved.

41. The method of claim 31, wherein ERG function is improved.

42. The method of claim 31, wherein inner retinal function is improved.

43. The method of claim 31, wherein photoreceptor function is improved.

44. A method for increasing survival of retinal structure in a subject in need thereof, comprising administering to said subject 10 to 200 mg per day of N-[(3S,4S)-6-acetyl-3-hydroxy-2,2-dimethyl-3,4-dihydrochromen-4-yl]-3-chloro-4-fluorobenzamide (Xiflam).

45. The method of claim 44, wherein said retinal structure comprises retinal pigment epithelium, retinal vascular endothelium, and/or retinal layer structure.

46. The method of claim 44, wherein micro- and/or macro-aneurysms in the retina are reduced.

47. A method for increasing survival of choroidal function in a subject in need thereof, comprising administering to said subject 10 to 200 mg per day of N-[(3S,4S)-6-acetyl-3-hydroxy-2,2-dimethyl-3,4-dihydrochromen-4-yl]-3-chloro-4-fluorobenzamide (Xiflam).

48. The method of claim 47, wherein choroidal blood flow is improved.

49. The method of claim 47, wherein choroidal vascular blood flow supplying the outer retina is improved.

50. The method of claim 47, wherein modulation of choroidal blood flow is improved.

51. A method for increasing survival of choroidal structure in a subject in need thereof, comprising administering to said subject 10 to 200 mg per day of N-[(3S,4S)-6-acetyl-3-hydroxy-2,2-dimethyl-3,4-dihydrochromen-4-yl]-3-chloro-4-fluorobenzamide (Xiflam).

52. The method of claim 51, wherein choroidal thickness is improved.

53. The method of claim 51, wherein the choroidal vascular bed is improved.

54. The method of claim 31, wherein increasing survival of retinal function is restoring or rescuing retinal function.

55. The method of claim 44, wherein increasing survival of retinal structure is restoring or rescuing retinal structure.

56. The method of claim 47, wherein increasing survival of choroidal function is restoring or rescuing choroidal function.

57. The method of claim 51, increasing survival of choroidal structure is restoring or rescuing choroidal structure.
